(19) United States
(12) Patent Application Publication
(10) Pub. No.: US 2026/0071279 A1
(43) Pub. Date: Mar. 12, 2026

(54) METHODS FOR IDENTIFYING CNS CANCER IN A SUBJECT

(71) Applicant: The Johns Hopkins University, Baltimore, MD (US)

(72) Inventors: Christopher Douville, Baltimore, MD (US); Chetan Bettegowda, Lutherville, MD (US); Bert Vogelstein, Baltimore, MD (US); Kenneth W. Kinzler, Frankford, DE (US); Nickolas Papadopoulos, Towson, MD (US)

(21) Appl. No.: 18/873,180

(22) PCT Filed: Jun. 8, 2023
(86) PCT No.: PCT/US2023/024846

§ 371 Date: Dec. 9, 2024

Related U.S. Application Data
(60) Provisional application No. 63/350,906, filed on Jun. 10, 2022.

(51) Int. Cl. C12Q 1/6886

(52) U.S. Cl. CPC C12Q 1/6886; C12Q 2600/156

(57) ABSTRACT

Provided herein are methods of identifying a subject as having a central nervous system (CNS) cancer that include (a) obtaining a DNA sample from the subject; (b) analyzing a plurality of chromosomal sequences in the DNA sample; (c) determining at least a portion of a nucleic acid sequence of one or more of the plurality of chromosomal sequences; (d) mapping the determined nucleic acid sequence to a reference chromosome; (e) dividing the DNA sample into a plurality of genomic intervals; (f) quantifying a plurality of features for the one or more nucleic acid sequences mapped to the genomic intervals; and (g) comparing the plurality of features in a first genomic interval with the plurality of features in one or more different genomic intervals and detecting a chromosomal abnormality in the DNA sample, thereby identifying the subject as having the CNS cancer.

### CROSS-REFERENCE TO RELATED APPLICATIONS

This application claims priority to U.S. Provisional Patent Application No. 63/350,906, filed on Jun. 10, 2022, which is incorporated herein by reference in its entirety.

### FEDERALLY SPONSORED RESEARCH OR DEVELOPMENT

This invention was made with government support under grant CA006973, CA230691, CA230400 and C208723 awarded by the National Institutes of Health. The government has certain rights in the invention.

### TECHNICAL FIELD

The present disclosure relates to the area of nucleic acid analysis. In particular, it relates to nucleic acid sequence analysis which can determine a chromosomal abnormality in a subject and identify the subject as having a central nervous system (CNS) cancer.

### BACKGROUND

Central nervous system (CNS) neoplasms represent a very heterogeneous class of tumors that are classified as primary, those originating in the brain or spinal cord, or metastatic, cancers that spread to the CNS from another organ. Approximately 24,500 cases of primary brain cancers occur a year in the United States, with the most common being glioblastoma in adults and medulloblastoma in children. Metastatic spread to the brain is very common, with 100,000 cases reported annually in the United States, with lung and breast being the most frequent. Cancers can spread to the brain matter itself, which are called parenchymal metastases (PM) or to the covering of the brain, also known as leptomeningeal disease (LMD). Unfortunately, regardless of primary vs metastatic, CNS cancers are a tremendous cause of morbidity and mortality, with few treatment strategies that lead to cure.

A pressing clinical challenge is the lack of any reliable biomarkers for the diagnosis and monitoring of cancers involving the CNS. The current gold standard is cytology on cerebrospinal fluid (CSF), which has a sensitivity as low as 2% depending on cancer type. In addition, to achieve maximum sensitivity, cytology requires large (>10 ml) volumes of CSF, necessitating in many cases multiple lumbar punctures. In addition, current imaging strategies with magnetic resonance imaging (MRI) cannot readily distinguish cancer from inflammatory or other non-neoplastic processes and can detect disease only after it has caused anatomic perturbations. Therefore, neurosurgical biopsy remains the exclusive means for diagnosing CNS neoplasms. Sole reliance on invasive procedures is suboptimal, as surgical intervention on the brain is fraught with risks including neurological injury, need of anesthesia and hospitalization and costs that are measured in the thousands of dollars per patient.

### SUMMARY

Provided herein are methods of identifying a subject as having a central nervous system (CNS) cancer, the method comprising: (a) obtaining a DNA sample from the subject; (b) analyzing a plurality of chromosomal sequences in the DNA sample; (c) determining at least a portion of a nucleic acid sequence of one or more of the plurality of chromosomal sequences; (d) mapping the determined nucleic acid sequence to a reference chromosome; (e) dividing the DNA sample into a plurality of genomic intervals; (f) quantifying a plurality of features for the one or more nucleic acid sequences mapped to the genomic intervals; and (g) comparing the plurality of features in a first genomic interval with the plurality of features in one or more different genomic intervals and detecting a chromosomal abnormality in the DNA sample, thereby identifying the subject as having the CNS cancer. In some embodiments, the subject is not known to have a CNS cancer.

Also provided herein are methods of monitoring a central nervous system (CNS) cancer in a subject, the method comprising: (a) obtaining a DNA sample from the subject; (b) analyzing a plurality of chromosomal sequences in the DNA sample; (c) determining at least a portion of a nucleic acid sequence of one or more of the plurality of chromosomal sequences; (d) mapping the determined nucleic acid sequence to a reference chromosome; (e) dividing the DNA sample into a plurality of genomic intervals; (f) quantifying a plurality of features for the one or more nucleic acid sequences mapped to the genomic intervals; (g) comparing the plurality of features in a first genomic interval with the plurality of features in one or more different genomic intervals and detecting a chromosomal abnormality in the DNA sample from the subject; and (h) repeating steps (a)-(g) at multiple time points, thereby monitoring progression of the CNS cancer in the subject. In some embodiments, the analyzing step (b) comprises amplifying the plurality of chromosomal sequences in the DNA sample with a pair of primers complementary to the plurality sequences to form a plurality of amplicons.

In some embodiments, the methods further comprise detecting the chromosomal abnormality in the DNA sample and identifying the chromosomal abnormality as a prognostic biomarker in the subject. In some embodiments, the chromosomal abnormality is selected from aneuploidy, a focal amplification, tumor mutation burden, chromosomal copy number changes, or cfDNA size. In some embodiments, the detection of chromosomal copy number changes is used to determine a type of cancer in the subject.

In some embodiments, the multiple time points comprise every week, every two weeks, every four weeks, every six weeks, or every eight weeks. In some embodiments, step (h) is performed at a time point after an anti-cancer treatment for the CNS cancer is administered to the subject. In some embodiments, step (h) further comprises determining minimal residual disease (MRD) in the subject. In some embodiments, the anti-cancer treatment comprises ionizing radiation, a chemotherapeutic agent, a therapeutic antibody, a checkpoint inhibitor, or any combination thereof.

In some embodiments, the DNA sample comprises at least 0.1 ng of DNA. In some embodiments, the DNA sample comprises tumor derived DNA. In some embodiments, the DNA sample is from a cerebrospinal fluid sample. In some embodiments, the DNA sample is obtained from the subject by lumbar puncture. In some embodiments, the DNA sample is from a blood plasma sample. In some embodiments, the DNA sample is obtained from the subject by venipuncture.

In some embodiments, an amplicon of the plurality of amplicons has a length of 100 basepairs or less. In some embodiments, an amplicon of the plurality of amplicons has a length of 200 basepairs or less. In some embodiments, the plurality of amplicons comprise nucleic acid sequences that can be mapped to a plurality of chromosomes.

In some embodiments, the CNS cancer is meningioma, pituitary adenoma, craniopharyngioma, neurofibroma, hemangioblastoma, encephalocele, fibrous dysplasia, glioma, astrocytomas, oligodendrogliomas, glioblastomas, ependymal tumors, hemangiopericytoma, germ cell tumors, chordoma, chondrosarcoma, medulloblastoma, olfactory neuroblastoma, lymphoma, gliosarcoma, rhabdomyosarcoma, paranasal sinus cancer, or atypical teratoid/rhabdoid tumor (AT/RT). In some embodiments, the CNS cancer is glioblastoma (GBM), medulloblastoma, parenchymal metastases (PM), leptomeningeal disease (LMD), diffuse large B-cell lymphoma, or CNS lymphoma.

In some embodiments, the obtaining step (a) comprises obtaining a first DNA sample and a second DNA sample from the subject. In some embodiments, the first DNA sample is a cerebrospinal fluid sample. In some embodiments, the second DNA sample is a blood plasma sample.

Also provided herein are methods of treating a CNS cancer in a subject in need thereof, the method comprising: (a) diagnosing the subject as having the CNS cancer according to any one of the claims 1-25; and (b) administering an anti-cancer treatment to the subject.

In some embodiments, the CNS cancer is meningioma, pituitary adenoma, craniopharyngioma, neurofibroma, hemangioblastoma, encephalocele, fibrous dysplasia, glioma, astrocytomas, oligodendrogliomas, glioblastomas, ependymal tumors, hemangiopericytoma, germ cell tumors, chordoma, chondrosarcoma, medulloblastoma, olfactory neuroblastoma, lymphoma, gliosarcoma, rhabdomyosarcoma, paranasal sinus cancer, or atypical teratoid/rhabdoid tumor (AT/RT). In some embodiments, the CNS cancer is glioblastoma (GBM), medulloblastoma, parenchymal metastases (PM), leptomeningeal disease (LMD), diffuse large B-cell lymphoma, or CNS lymphoma. In some embodiments, the anti-cancer treatment comprises ionizing radiation, a chemotherapeutic agent, a therapeutic antibody, a checkpoint inhibitor, or any combination thereof.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 shows an overview of the RealSeqS-CNS approach: Using a single PCR primer to concomitantly amplify ˜350,000 Alu SINE elements spread throughout the genome, RealSeqS-CNS uses supervised machine learning to combine large scale chromosome aneuploidies with known focal changes in cancer of the Central Nervous System and mutation burden to detect the presence of cancer.

FIGS. 2A-2D show representative Focal Changes used in RealSeqS-CNS. The RealSeqS-CNS Focal panel calls focal changes surrounding the following genes: FIG. 2A: MDM4; FIG. 2B: EGFR; FIG. 2C: CDK4.; and FIG. 2D: ERBB2.

FIGS. 3A-3E show evaluation of RealSeqS-CNS: FIG. 3A: Comparison of performance of RealSeqS-CNS in the Training and Validation Partitions; FIG. 3B: Heatmap of the different components of the classifier; FIG. 3C: Comparison of Performance of RealSeqS-CNS to Cytology;

FIG. 3D: Decision Tree for the Molecular Classification of Positive CNS Cancers; and FIG. 3E: Comparison of Performance of RealSeqS-CNS in CSF and Plasma.

FIGS. 4A-4D show estimation of the size of CSF DNA using RealSeqS. RealSeqS loci range from 70-500 base pairs (bps) with most amplicons ranging from 80-85 bps. The probability of short and long fragments were evaluated and how it pertains to both cancer status as well as the RealSeqS-CNS classification. FIG. 4A: The distribution of the empirical Probability Mass Function (ePMF) for plasma and CSF; FIG. 4B: Probability of observing small loci (<200 bps) for Non-cancer and Cancer CSF samples; FIG. 4C: Probability of observing small loci (<200 bps) compared to RealSeqS-CNS call; and FIG. 4D: Probability of observing small loci (<200 bps) for each cancer type.

### DETAILED DESCRIPTION

While there are no routinely used biomarkers for central nervous system cancers, there have been several exciting putative biomarkers proposed for CNS tumors. Given the relative lack of shedding of tumor derived material into the circulation, CSF has become an appealing biofluid to explore given the elevated levels of tumor derived DNA (CSF-tDNA) and other markers such as tumor derived RNA and proteins. While CSF sampling is more invasive than venipuncture, it is already a part of standard of care for several CNS neoplasms including medulloblastoma, LMD and central nervous system lymphomas (CNSL). In CNSL, cerebrospinal fluid is routinely sent for cytology and flow cytometry. However, the sensitivity is less than 50% but in those cases where a diagnosis can be established, patients can proceed directly to chemotherapy and radiation therapy and bypass a surgical biopsy. Unfortunately, the performance of conventional testing is so poor that the majority of patients are still required to undergo neurosurgical procedures despite CSF sampling. A reliable diagnostic biomarker could circumvent the need for a biopsy in patients with CNSL.

While CSF-tDNA is an attractive analyte for biomarker development, it poses several challenges. Foremost among these is the large heterogeneity of cancers that arise and affect the brain. Each cancer type has unique and distinct somatic mutation profiles, making the development of a multi-cancer detection strategy difficult. In addition, the quantity of total DNA found in the CSF is often trace, making approaches that require large quantities of starting material problematic. The most sensitive CSF-tDNA detection strategies reported to date utilize a personalized approach, where the tumor tissue is already available and genotype known.

Provided herein are methods of identifying a subject as having a central nervous system (CNS) cancer that include: (a) obtaining a DNA sample from the subject; (b) analyzing a plurality of chromosomal sequences in the DNA sample; (c) determining at least a portion of a nucleic acid sequence of one or more of the plurality of chromosomal sequences; (d) mapping the determined nucleic acid sequence to a reference chromosome; (e) dividing the DNA sample into a plurality of genomic intervals; (f) quantifying a plurality of features for the one or more nucleic acid sequences mapped to the genomic intervals; and (g) comparing the plurality of features in a first genomic interval with the plurality of features in one or more different genomic intervals and detecting a chromosomal abnormality in the DNA sample, thereby identifying the subject as having the CNS cancer.

Also provided herein are methods of monitoring a central nervous system (CNS) cancer in a subject that include: (a) obtaining a DNA sample from the subject; (b) analyzing a plurality of chromosomal sequences in the DNA sample; (c) determining at least a portion of a nucleic acid sequence of one or more of the plurality of chromosomal sequences; (d) mapping the determined nucleic acid sequence to a reference chromosome; (e) dividing the DNA sample into a plurality of genomic intervals; (f) quantifying a plurality of features for the one or more nucleic acid sequences mapped to the genomic intervals; (g) comparing the plurality of features in a first genomic interval with the plurality of features in one or more different genomic intervals and detecting a chromosomal abnormality in the DNA sample from the subject; and (h) repeating steps (a)-(g) at multiple time points, thereby monitoring progression of the CNS cancer in the subject.

Various non-limiting aspects of these methods are described herein, and can be used in any combination without limitation. Additional aspects of various components of methods for identifying the presence or absence of a chromosomal abnormality are known in the art.

It must be noted that, as used in the specification and the appended claims, the singular forms “a,” “an” and “the” include plural referents unless the context clearly dictates otherwise.

As used herein, the term “about”, when used in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that are within 25%, 20%, 19%, 18%, 17%, 16%, 15%, 14%, 13%, 12%, 11%, 10%, %, 8%, 7%, 6%, 5%, 4%, 3%, 2%, 1%, or less of the referred value.

As used herein, the term “biological sample” refers to a sample obtained from a subject for analysis using any of a variety of techniques including, but not limited to, biopsy, surgery, and laser capture microscopy (LCM), and generally includes cells and/or other biological material from the subject. A biological sample can be obtained from a eukaryote, such as a patient derived organoid (PDO) or patient derived xenograft (PDX). The biological sample can include organoids, a miniaturized and simplified version of an organ produced in vitro in three dimensions that shows realistic micro-anatomy. Subjects from which biological samples can be obtained can be healthy or asymptomatic individuals, individuals that have or are suspected of having a disease (e.g., cancer) or a pre-disposition to a disease, and/or individuals that are in need of therapy or suspected of needing therapy. In some embodiments, biological samples can include one or more diseased cells. A diseased cell can have altered metabolic properties, gene expression, protein expression, and/or morphologic features. Examples of diseases include inflammatory disorders, metabolic disorders, nervous system disorders, and cancer.

The biological sample can include any number of macromolecules, for example, cellular macromolecules and organelles (e.g., mitochondria and nuclei). In some embodiments, the biological sample can be a nucleic acid sample and/or protein sample. In some embodiments, the biological sample can be a carbohydrate sample or a lipid sample. The biological sample can be obtained as a tissue sample, such as a tissue section, biopsy, a core biopsy, needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions.

As used herein, biological samples can include but are not limited to plasma, serum, blood, tissue, tumor sample, stool, sputum, saliva, urine, sweat, tears, ascites, bronchoaveolar lavage, semen, archeologic specimens and forensic samples. In some embodiments, the biological sample is a solid biological sample (e.g., a tumor sample). In some embodiments, the biological sample is a liquid biological sample. Liquid biological samples can include, but are not limited to plasma, serum, blood, sputum, saliva, urine, sweat, tears, ascites, bronchoaveolar lavage, and semen. In some embodiments, the liquid biological sample is cell free or substantially cell free. In some embodiments, the biological sample is a plasma or serum sample. In some embodiments, the liquid biological sample is a whole blood sample. In some embodiments, the liquid biological sample comprises peripheral mononuclear blood cells. In some embodiments, the biological sample is a cerebrospinal fluid (CSF) sample.

As used herein, the terms “cancer”, “malignancy”, “neoplasm”, “tumor”, and “carcinoma”, refer to cells that exhibit relatively abnormal, uncontrolled, and/or autonomous growth, so that they exhibit an aberrant growth phenotype characterized by a significant loss of control of cell proliferation. In some embodiments, a tumor may be or comprise cells that are precancerous (e.g., benign), malignant, pre-metastatic, metastatic, and/or non-metastatic. The present disclosure specifically identifies certain cancers to which its teachings may be particularly relevant. In some embodiments, a relevant cancer may be characterized by a solid tumor. In some embodiments, a relevant cancer may be characterized by a hematologic tumor. In general, examples of different types of cancers known in the art include, for example, hematopoietic cancers including leukemias, lymphomas (Hodgkin's and non-Hodgkin's), myelomas and myeloproliferative disorders; sarcomas, melanomas, adenomas, carcinomas of solid tissue, squamous cell carcinomas of the mouth, throat, larynx, and lung, liver cancer, genitourinary cancers such as prostate, cervical, bladder, uterine, and endometrial cancer and renal cell carcinomas, bone cancer, pancreatic cancer, skin cancer, cutaneous or intraocular melanoma, cancer of the endocrine system, cancer of the thyroid gland, cancer of the parathyroid gland, head and neck cancers, breast cancer, gastro-intestinal cancers and nervous system cancers, benign lesions such as papillomas, and the like. In some embodiments, a cancer is a central nervous system (CNS) cancer. As used herein, the term “central nervous system cancer” refers to a cancer in which abnormal cells form in the tissues of the brain and/or spinal cord. In some embodiments, a CNS cancer is a primary brain tumor, wherein the tumor starts in the brain. In some embodiments, the primary CNS cancer can start from brain cells, membranes around the brain (e.g., meninges), nerves, or the glands. In some embodiments, a CNS cancer is a metastatic CNS cancer (e.g., secondary brain tumors), wherein the cancer is caused by cancer cells that spread (e.g., metastasizing) to the brain from a different part of the body. In some embodiments, a cancer that can spread to the brain can include lung cancer, breast cancer, skin (melanoma) cancer, colon cancer, kidney cancer, and thyroid gland cancer. In some embodiments, a CNS cancer can include meningioma, pituitary adenoma, craniopharyngioma, neurofibroma, hemangioblastoma, encephalocele, fibrous dysplasia, glioma, astrocytomas, oligodendrogliomas, glioblastomas, ependymal tumors, hemangiopericytoma, germ cell tumors, chordoma, chondrosarcoma, medulloblastoma, olfactory neuroblastoma, lymphoma, gliosarcoma, rhabdomyosarcoma, paranasal sinus cancer, and atypical teratoid/rhabdoid tumor (AT/RT).

As used herein, “nucleic acid” is used to refer to any compound and/or substance that comprise a polymer of nucleotides. In some embodiments, a polymer of nucleotides are referred to as polynucleotides. Exemplary nucleic acids or polynucleotides can include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-a-LNA having a 2′-amino functionalization) or hybrids thereof. Naturally-occurring nucleic acids generally have a deoxyribose sugar (e.g., found in deoxyribonucleic acid (DNA)) or a ribose sugar (e.g., found in ribonucleic acid (RNA)). A nucleic acid can contain nucleotides having any of a variety of analogs of these sugar moieties that are known in the art. A deoxyribonucleic acid (DNA) can have one or more bases selected from the group consisting of adenine (A), thymine (T), cytosine (C), or guanine (G), and a ribonucleic acid (RNA) can have one or more bases selected from the group consisting of uracil (U), adenine (A), cytosine (C), or guanine (G).

In some embodiments, the term “nucleic acid” refers to a deoxyribonucleic acid (DNA) or ribonucleic acid (RNA), or a combination thereof, in either a single- or double-stranded form. Unless specifically limited, the term encompasses nucleic acids containing known analogues of natural nucleotides that have similar binding properties as the reference nucleotides. Unless otherwise indicated, a particular nucleic acid sequence also implicitly encompasses complementary sequences as well as the sequence explicitly indicated. In some embodiments of any of the isolated nucleic acids described herein, the isolated nucleic acid is DNA. In some embodiments of any of the isolated nucleic acids described herein, the isolated nucleic acid is RNA.

As used herein, the term “subject” is intended to refer to any subject. In some embodiments, the subject is cat, a dog, a goat, a human, a non-human primate, a rodent (e.g., a mouse or a rat), a pig, or a sheep. In some embodiments, a subject is suffering from a relevant disease, disorder or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

### Method of Identifying Central Nervous System (CNS) Cancer in a Subject

Provided herein are methods of identifying a subject as having a central nervous system (CNS) cancer that include (a) obtaining a DNA sample from the subject; (b) analyzing a plurality of chromosomal sequences in the DNA sample; (c) determining at least a portion of a nucleic acid sequence of one or more of the plurality of chromosomal sequences; (d) mapping the determined nucleic acid sequence to a reference chromosome; (e) dividing the DNA sample into a plurality of genomic intervals; (f) quantifying a plurality of features for the one or more nucleic acid sequences mapped to the genomic intervals; and (g) comparing the plurality of features in a first genomic interval with the plurality of features in one or more different genomic intervals and detecting a chromosomal abnormality in the DNA sample, thereby identifying the subject as having the CNS cancer. In some embodiments, the subject is not known to have a CNS cancer.

In some embodiments, the analyzing step (b) comprises amplifying the plurality of chromosomal sequences in the DNA sample with a pair of primers complementary to the plurality sequences to form a plurality of amplicons.

As used herein, a “DNA sample” can refer to a biological sample that includes DNA. In some embodiments, the DNA sample includes about 0.05 ng to about 0.3 ng (e.g., about 0.05 ng to about 0.25 ng, about 0.05 ng to about 0.2 ng, about 0.05 ng to about 0.15 ng, about 0.05 ng to about 0.1 ng, about 0.1 ng to about 0.3 ng, about 0.1 ng to about 0.25 ng, about 0.1 ng to about 0.2 ng, about 0.1 ng to about 0.15 ng, about 0.15 ng to about 0.3 ng, about 0.15 ng to about 0.25 ng, about 0.15 ng to about 0.2 ng, about 0.2 ng to about 0.3 ng, about 0.2 ng to about 0.25 ng, or about 0.25 ng to about 0.3 ng) of DNA. In some embodiments, the DNA sample includes about 0.1 ng to about 0.25 ng of DNA. In some embodiments, the DNA sample comprises at least 0.1 ng of DNA. In some embodiments, the DNA sample can include tumor derived DNA. In some embodiments, the DNA sample can include cell-free circulating DNA (e.g., cell-free circulating fetal DNA). In some embodiments, the DNA sample can include circulating tumor DNA (ctDNA).

In some embodiments, the DNA sample is from a cerebrospinal fluid sample. In some embodiments, the DNA sample is obtained from the subject by lumbar puncture. In some embodiments, the cerebrospinal fluid sample is obtained by surgical aspiration, ventricular catheter, or radiology guided CSF sampling. In some embodiments, the DNA sample is from a cerebrospinal fluid sample that is about 0.25 ml to about 2.0 ml (e.g., about 0.25 ml to about 1.5 ml, about 0.25 ml to about 1.0 ml, about 0.25 ml to about 0.75 ml, about 0.25 ml to about 0.5 ml, about 0.5 ml to 2.0 ml, about 0.5 ml to about 1.5 ml, about 0.5 ml to about 1.0 ml, about 0.5 ml to about 0.75 ml, about 0.75 ml to 2.0 ml, about 0.75 ml to about 1.5 ml, about 0.75 ml to about 1.0 ml, about 0.1 ml to 2.0 ml, about 0.1 ml to about 1.5 ml, or about 1.5 ml to about 2.0 ml) in volume. In some embodiments, the DNA sample is from a cerebrospinal fluid sample that is about 0.5 ml to about 1.0 ml. In some embodiments, the DNA sample is from a blood plasma sample. In some embodiments, the DNA sample is obtained from the subject by venipuncture. In some embodiments, the DNA sample is from a biological sample of 1 mL or less in volume (e.g., about 1 ml, about 0.9 ml, about 0.8 ml, about 0.7 ml, about 0.6 ml, about 0.5 ml, about 0.4 ml, about 0.3 ml, about 0.2 ml, or about 0.1 ml).

In some embodiments, a nucleic acid sample (e.g., cfDNA) has been isolated and purified from the biological sample. Nucleic acid can be isolated and purified from the biological sample using any means known in the art. For example, a biological sample may be processed to separate nucleic acids from unwanted components of the biological sample (e.g., proteins, cell walls, other contaminants). For example, nucleic acid can be extracted from the biological sample using liquid extraction (e.g., Trizol, DNAzol) techniques. Nucleic acid can also be extracted using commercially available kits (e.g., Qiagen DNeasy kit, QIAamp kit, Qiagen Midi kit, QIAprep spin kit).

In some embodiments, the methods described herein can be used to identify a subject as having a disease. In some embodiments, the disease is a cancer. In some embodiments, the cancer is a cancer of the central nervous system (CNS). In some embodiments, a CNS cancer is a primary brain tumor. In some embodiments, a CNS cancer is a metastatic CNS cancer (e.g., secondary brain tumors). In some embodiments, a CNS cancer can include meningioma, pituitary adenoma, craniopharyngioma, neurofibroma, hemangioblastoma, encephalocele, fibrous dysplasia, glioma, astrocytomas, oligodendrogliomas, glioblastomas, ependymal tumors, hemangiopericytoma, germ cell tumors, chordoma, chondrosarcoma, medulloblastoma, olfactory neuroblastoma, lymphoma, gliosarcoma, rhabdomyosarcoma, paranasal sinus cancer, and atypical teratoid/rhabdoid tumor (AT/RT). In some embodiments, the CNS cancer can include a glioblastoma (GBM), medulloblastoma, parenchymal metastases (PM), leptomeningeal disease (LMD), diffuse large B-cell lymphoma, or CNS lymphoma.

### Detecting a Chromosomal Abnormality

As used herein, a “chromosomal abnormality” or “chromosomal anomaly” refers to a change in the genetic material or DNA in a subject. A chromosomal abnormality can result from a change in the number or structure of chromosomes. A numerical abnormality are caused by the loss or gain of whole chromosomes, which can affect hundreds, or even thousands of genes. A structural abnormality is caused when large sections of DNA are missing from or are added to a chromosome. In some embodiments, a structural abnormality can be caused by a deletion mutation, duplication mutation, translocation mutation, or inversion mutation. In some embodiments, a chromosomal abnormality can include aneuploidy, focal amplification, tumor mutation burden, or difference in cfDNA size.

Provides herein are methods and materials for identifying one or more chromosomal abnormalities (e.g., aneuploidies, focal amplification, tumor mutation burden, or difference in cfDNA size) in a sample. In some embodiments, methods and materials described herein are used to identify one or more chromosomal abnormalities (e.g., aneuploidies, focal amplification, tumor mutation burden, or difference in cfDNA size) in a subject (e.g., a juvenile subject or an adult subject). For example, a subject (e.g., a sample obtained from a subject) can be assessed for the presence or absence of one or more chromosomal abnormalities. In some embodiments, the methods and materials provided herein can use amplicon-based sequencing data to identify a subject as having a disease associated with one or more chromosomal abnormalities (e.g., cancer). For example, methods and materials described herein can be applied to a sample obtained from a subject to identify the subject as having one or more chromosomal abnormalities. For example, methods and materials described herein can be applied to a sample obtained from a subject to identify the subject as having a disease associated with one or more chromosomal abnormalities (e.g., cancer). This document also provides methods and materials for identifying and/or treating a disease or disorder associated with one or more chromosomal abnormalities (e.g., one or more chromosomal abnormalities identified as described herein). In some cases, one or more chromosomal abnormalities can be identified in DNA (e.g., genomic DNA) obtained from a sample obtained from a subject. In some embodiments, a subject identified as having cancer based, at least in part, on the presence of one or more chromosomal abnormalities can be treated with one or more cancer treatments.

Also disclosed herein, are methods of increasing the sensitivity of detecting one or more cancers, or a plurality of cancers, without altering the specificity of detecting said cancer or a plurality of cancers. In an embodiment, the sensitivity of detection of a cancer by evaluating (i) a genetic biomarker, e.g. a somatic mutation; (ii) a protein biomarker; and (iii) presence of a chromosomal abnormality, is higher, e.g., about 1.1, 1.2, 1.3, 1.4, 1.5, 2, 2.5, 3, 3.5, 4, 4.5, 5, 5.5, 6, 6.5, 7, 7.5, 8, 8.5, 9, 9.5, or 10 fold higher, than the sensitivity of detection of the cancer by evaluating (i) alone; (ii) alone; (iii) alone; (i) and (ii) only; (i) and (iii) only; or (ii) and (iii) only. The increase in sensitivity by a method comprising (i), (ii) and (iii) does not alter, e.g., reduce the specificity of detecting the cancer, or plurality of cancers. In some embodiments, the methods described herein can include evaluating the presence of a chromosomal abnormality from one or more DNA samples, or a plurality of DNA samples. In some embodiments, a method described herein includes the obtaining step (a), wherein the obtaining step (a) comprises obtaining a first DNA sample and a second DNA sample from the subject. In some embodiments, first DNA sample is a cerebrospinal fluid sample. In some embodiments, the second DNA sample is a blood plasma sample. In some embodiments, the methods described herein can increase the sensitivity of detecting one or more cancers by evaluating the presence of a chromosomal abnormality from one or more DNA samples, or a plurality of DNA samples.

In some embodiments, methods described herein can include amplification of a plurality of amplicons. In some embodiments, the plurality of amplicons is amplified from a plurality of chromosomal sequences in a DNA sample. In some embodiments, the plurality of amplicons is amplified with a pair of primers complementary to the plurality of chromosomal sequences. In some embodiments, the plurality of amplicons can be amplified from any variety of repetitive elements. In some embodiments, the plurality of amplicons is amplified from a plurality of short interspersed nucleotide elements (SINEs). In some embodiments, the plurality of amplicons is amplified from a plurality of long interspersed nucleotide elements (LINEs). Methods of amplifying a plurality of amplicons include, without limitation, the polymerase chain reaction (PCR) and isothermal amplification methods (e.g., rolling circle amplification or bridge amplification). In some embodiments, a second amplification step is performed. In some embodiments, the amplified DNA from a first amplification reaction is used as a template in a second amplification reaction. In some embodiments, the amplified DNA is purified before the second amplification reaction (e.g., PCR purification using methods known in the art).

In some embodiments, a first primer comprises from the 5′ to 3′ end: a universal primer sequence (UPS), a unique identifier DNA sequence (UID), and an amplification sequence. In some embodiments, the first primer comprises from the 5′ to 3′ end: a UPS sequence and an amplification sequence. In some embodiments, the first primer comprises from the 5′ to 3′ end: an amplification sequence. In such cases in which the first primer comprises at least an amplification sequence, any variety of library generation techniques known in the art can be used to generate a next generation sequencing library from the amplified amplicons. In some embodiments, the UID comprises a sequence of 16-20 degenerate bases. In some embodiments, a degenerate sequence is a sequence in which some positions of a nucleotide sequence contain a number of possible bases. In some embodiments of any of the methods described herein, a degenerate sequence can be a degenerate nucleotide sequence comprising about or at least 5, 6, 7, 8, 9, 10, 11, 12, 13, 14, 15, 16, 17, 18, 19, 20, 25, 30, 35, 40, 45, or 50 nucleotides. In some embodiments, a nucleotide sequence contains 1, 2, 3, 4, 5, 6, 7, 8, 9, 0, 10, 15, 20, 25, or more degenerate positions within the nucleotide sequence. In some embodiments, the degenerate sequence is used as a unique identifier DNA sequence (UID). In some embodiments, the degenerate sequence is used to improve the amplification of an amplicon. For example, a degenerate sequence may contain bases complementary to a chromosomal sequence being amplified. In such cases, the increased complementarity may increase a primers affinity for the chromosomal sequence. In some embodiments, the UID (e.g., degenerate bases) is designed to increase a primers affinity to a plurality of chromosomal sequences.

In some embodiments, an amplification reaction includes one or more pairs of primers. In some embodiments, an amplification reaction includes at least 1, at least 2, at least 3, at least 4, at least 5, at least 6, at least 7, at least 8, or at least 9 pairs of primers.

In some embodiments, a pair of primers (e.g., a single pair of primers) are complementary to a plurality of chromosomal sequences. As used herein, the term “complementary” or “complementarity” refers to nucleic acid residues that are capable or participating in Watson-Crick type or analogous base pair interactions that is enough to support amplification. In some embodiments, an amplification sequence of a first primer is designed to amplify one or more chromosomal sequences. In some embodiments, the one or more chromosomal sequence include any of a variety of repetitive elements as described herein. In some embodiments, the chromosomal sequences are SINEs. In some embodiments, the chromosomal sequences are LINEs. In some embodiments, the chromosomal sequences are a mixture of different types of repetitive elements (e.g., SINEs, LINEs and/or other exemplary repetitive elements). In some embodiments when an amplification reaction includes two or more pairs of primers, each pair of primers amplifies a different type of repetitive element. For example, a first pair of primers can amplify SINEs, and a second pair of primers can amplify LINEs. Optionally, a third, fourth, fifth, etc. pair of primers can amplify a third, fourth, fifth, etc. type of repetitive element. In some embodiments when an amplification reaction includes two or more pairs of primers, each pair of primers generates amplicons from the same type of repetitive element. For example, a first pair of primers can amplify SINEs, and a second pair of primers amplify SINEs. Optionally, a third, fourth, fifth, etc. pair of primers can amplify SINEs. In some embodiments when an amplification reaction includes two or more primer pairs, each pair of primers generates amplicons from a mixture of different types of repetitive elements.

In some embodiments, methods described herein include using amplicon-based sequencing reads. In some embodiments, a plurality of amplicons (e.g., amplicons obtained from a DNA sample) are sequenced. In some embodiments, each amplicon is sequenced at least 1, 2, 3, 4, 5, 6, 7, 8, 9, 10, 11, 12, 13, 14, 15, 16, 17, 18, 19, 20, or more times. In some embodiments, each amplicon can be sequenced between about 1 and about 20 (e.g., between about 1 and about 15, between about 1 and about 12, between about 1 and about 10, between about 1 and about 8, between about 1 and about 5, between about 5 and about 20, between about 7 and about 20, between about 10 and about 20, between about 13 and about 20, between about 3 and about 18, between about 5 and about 16, or between about 8 and about 12) times. In some cases, amplicon-based sequencing reads can include continuous sequencing reads. In some cases, amplicons include short interspersed nucleotide elements (SINEs). In some cases, amplicon-based sequencing reads can include from about 100,000 to about 25 million (e.g., from about 100,000 to about 20 million, from about 100,000 to about 15 million, from about 100,000 to about 12 million, from about 100,000 to about 10 million, from about 100,000 to about 5 million, from about 100,000 to about 1 million, from about 100,000 to about 750,000, from about 100,000 to about 500,000, from about 100,000 to about 250,000, from about 250,000 to about 25 million, from about 250,000 to about 20 million, from about 250,000 to about 15 million, from about 250,000 to about 12 million, from about 250,000 to about 10 million, from about 250,000 to about 5 million, from about 250,000 to about 1 million, from about 250,000 to about 750,000, from about 250,000 to about 500,000, from about 500,000 to about 25 million, from about 500,000 to about 20 million, from about 500,000 to about 15 million, from about 500,000 to about 12 million, from about 500,000 to about 10 million, from about 500,000 to about 5 million, from about 500,000 to about 1 million, from about 500,000 to about 750,000, from about 750,000 to about 25 million, from about 750,000 to about 20 million, from about 750,000 to about 15 million, from about 750,000 to about 12 million, from about 750,000 to about 10 million, from about 750,000 to about 5 million, from about 750,000 to about 1 million, from about 1 million to about 25 million, from about 1 million to about 20 million, from about 1 million to about 15 million, from about 1 million to about 12 million, from about 1 million to about 10 million, from about 1 million to about 5 million, from about 1 million to about 25 million, from about 5 million to about 25 million, from about 5 million to about 20 million, from about 5 million to about 15 million, from about 5 million to about 12 million, from about 5 million to about 10 million, from about 10 million to about 25 million, from about 10 million to about 20 million, from about 10 million to about 15 million, from about 10 million to about 12 million, from about 12 million to about 25 million, from about 12 million to about 20 million, from about 12 million to about 15 million, from about 15 million to about 25 million, from about 15 million to about 20 million, or from about 20 million to about 25 million) sequencing reads. For example, sequencing a plurality of amplicons can include assigning a unique identifier (UID) to each template molecule (e.g., to each amplicon), amplifying each uniquely tagged template molecule to create UID-families, and redundantly sequencing the amplification products. For example, sequencing a plurality of amplicons can include calculating a Z-score of a variant on said selected chromosome arm using the equation

$$Z \sim \frac{{\sum }_{i = 1}^{k} {w}_{i} {Z}_{i}}{\sqrt{{\sum }_{i = 1}^{k} {w}_{i}^{2}}} ,$$

where wᵢ is UID depth at a variant i, Zᵢ is the Z-score of variant i, and k is the number of variants observed on the chromosome arm. In some embodiments, methods of sequencing amplicons includes methods known in the art (see, e.g., US Pat. No. 2015/0051085; and Kinde et al. 2012 PloS ONE 7:e41162, which are herein incorporated by reference in their entireties). In some embodiments, amplicons are aligned to a reference genome (e.g., GRC37).

In some embodiments, a plurality of amplicons generated by methods described herein includes from about 10,000 to about 1,000,000 (e.g., from about 15,000 to about 1,000,000, from about 25,000 to about 1,000,000, from about 35,000 to about 1,000,000, from about 50,000 to about 1,000,000, from about 75,000 to about 1,000,000, from about 100,000 to about 1,000,000, from about 125,000 to about 1,000,000, from about 160,000 to about 1,000,000, from about 180,000 to about 1,000,000, from about 200,000 to about 1,000,000, from about 300,000 to about 1,000,000, from about 500,000 to about 1,000,000, from about 750,000 to about 1,000,000, about 10,000 to about 750,000, from about 15,000 to about 750,000, from about 25,000 to about 750,000, from about 35,000 to about 750,000, from about 50,000 to about 750,000, from about 75,000 to about 750,000, from about 100,000 to about 750,000, from about 125,000 to about 750,000, from about 160,000 to about 750,000, from about 180,000 to about 750,000, from about 200,000 to about 750,000, from about 300,000 to about 750,000, from about 500,000 to about 750,000, about 10,000 to about 500,000, from about 15,000 to about 500,000, from about 25,000 to about 500,000, from about 35,000 to about 500,000, from about 50,000 to about 500,000, from about 75,000 to about 500,000, from about 100,000 to about 500,000, from about 125,000 to about 500,000, from about 160,000 to about 500,000, from about 180,000 to about 500,000, from about 200,000 to about 500,000, from about 300,000 to about 500,000, about 10,000 to about 300,000, from about 15,000 to about 300,000, from about 25,000 to about 300,000, from about 35,000 to about 300,000, from about 50,000 to about 300,000, from about 75,000 to about 300,000, from about 100,000 to about 300,000, from about 125,000 to about 300,000, from about 160,000 to about 300,000, from about 180,000 to about 300,000, from about 200,000 to about 300,000, about 10,000 to about 200,000, from about 15,000 to about 200,000, from about 25,000 to about 200,000, from about 35,000 to about 200,000, from about 50,000 to about 200,000, from about 75,000 to about 200,000, from about 100,000 to about 200,000, from about 125,000 to about 200,000, from about 160,000 to about 200,000, from about 180,000 to about 200,000, about 10,000 to about 180,000, from about 15,000 to about 180,000, from about 25,000 to about 180,000, from about 35,000 to about 180,000, from about 50,000 to about 180,000, from about 75,000 to about 180,000, from about 100,000 to about 180,000, from about 125,000 to about 180,000, from about 160,000 to about 180,000, about 10,000 to about 160,000, from about 15,000 to about 160,000, from about 25,000 to about 160,000, from about 35,000 to about 160,000, from about 50,000 to about 160,000, from about 75,000 to about 160,000, from about 100,000 to about 160,000, from about 125,000 to about 160,000, about 10,000 to about 125,000, from about 15,000 to about 125,000, from about 25,000 to about 125,000, from about 35,000 to about 125,000, from about 50,000 to about 125,000, from about 75,000 to about 125,000, from about 100,000 to about 125,000, about 10,000 to about 100,000, from about 15,000 to about 100,000, from about 25,000 to about 100,000, from about 35,000 to about 100,000, from about 50,000 to about 100,000, from about 75,000 to about 100,000, about 10,000 to about 75,000, from about 15,000 to about 75,000, from about 25,000 to about 75,000, from about 35,000 to about 75,000, from about 50,000 to about 75,000, about 10,000 to about 50,000, from about 15,000 to about 50,000, from about 25,000 to about 50,000, from about 35,000 to about 50,000, about 10,000 to about 35,000, from about 15,000 to about 35,000, from about 25,000 to about 35,000, about 10,000 to about 25,000, from about 15,000 to about 25,000, or about 10,000 to about 15,000) amplicons (e.g., unique amplicons).

Amplicons in a plurality of amplicons can include from about 50 to about 500 (e.g., about 50 to about 450, about 50 to about 400, about 50 to about 350, about 50 to about 300, about 50 to about 250, about 50 to about 200, about 50 to about 150, about 50 to about 100, about 100 to about 500, about 100 to about 450, about 100 to about 400, about 100 to about 350, about 100 to about 300, about 100 to about 250, about 100 to about 200, about 100 to about 150, about 150 to about 500, about 150 to about 450, about 150 to about 400, about 150 to about 350, about 150 to about 300, about 150 to about 250, about 150 to about 200, about 200 to about 500, about 200 to about 450, about 200 to about 400, about 200 to about 350, about 200 to about 300, about 200 to about 250, about 250 to about 500, about 250 to about 450, about 250 to about 400, about 250 to about 350, about 250 to about 300, about 300 to about 500, about 300 to about 450, about 300 to about 400, about 300 to about 350, about 350 to about 500, about 350 to about 450, about 350 to about 400, about 400 to about 500, about 400 to about 450, or about 450 to about 500) base pairs. In some embodiments, an amplicon can include about 80 to about 85 base pairs. In some embodiments, an amplicon of the plurality of amplicons has a length of 100 base pairs or less. In some embodiments, an amplicon of the plurality of amplicons has a length of 200 base pairs or less.

In some embodiments, one or more amplicons in a plurality of amplicons generated by methods described herein can be greater than 1000 basepairs (bp) in length (“long amplicons”). In some embodiments, one or more long amplicons make up at least 4.0% of all amplicons within the total plurality of amplicons. In some embodiments, methods and materials described herein can detect long amplicons when the long amplicons make up at least 4.0% of all the amplicons within the total plurality of amplicons. In some embodiments, methods and materials described herein can detect long amplicons when the long amplicons make up between 0.01% and 3.9% of all amplicons within the total plurality of amplicons.

In some embodiments, one or more amplicons with a length>1000 bp originate from amplification of DNA from cells that do not contain a chromosomal abnormality. In some embodiments, cells that do not contain chromosomal abnormalities are considered contaminating cells. In some embodiments, cells that do not contain chromosomal abnormalities are used as control cells or samples. In some embodiments, contaminating cells can be any variety of cells that might be found in a plasma sample that may dilute amplification of the intended target. In some embodiments, contaminating cells are white blood cells (e.g., leukocyte, granulocyte, eosinophil, basophile, B-cell, T-cell or Natural Killer cell). For example, contaminating cells can be leukocytes.

In some embodiments, methods described herein include grouping sequencing reads (e.g., from a plurality of amplicons) into clusters (e.g., unique clusters) of genomic intervals. In some embodiments, a genomic interval is included in one or more clusters. In some embodiments, a genomic interval can belong to from about 100 to about 252 (e.g., about 100 to about 225, about 100 to about 200, about 100 to about 175, about 100 to about 150, about 100 to about 125, about 125 to about 252, about 125 to about 225, about 125 to about 200, about 125 to about 175, about 125 to about 150, about 150 to about 252, about 150 to about 225, about 150 to about 200, about 150 to about 175, about 175 to about 252, about 175 to about 225, about 175 to about 200, about 200 to about 252, about 200 to about 225, or about 225 to about 252) clusters. In some embodiments, each cluster includes any appropriate number of genomic intervals. In some embodiments, each cluster includes the same number of genomic intervals. In some embodiments, different clusters include varying numbers of genomic clusters.

In some embodiments, genomic intervals are identified as having shared amplicon features. As used herein, the term “shared amplicon feature” refers to amplicons with one or more features that are similar. In some embodiments, a plurality of genomic intervals are grouped into a cluster based on one or more shared amplicon features of the sequencing reads mapped to a genomic interval. In some embodiments, the shared amplicon feature is the number amplicons mapped to a genomic interval (e.g., sums of the distributions of the sequencing reads in each genomic interval). In some embodiments, the shared amplicon feature is the average length of the mapped amplicons. In some embodiments, a plurality of amplicons comprise nucleic acid sequences that can be mapped to a plurality of chromosomes.

In some embodiments, a cluster of genomic intervals includes from about 5000 to about 6000 (e.g., from about 5000 to about 5800, from about 5000 to about 5600, from about 5000 to about 5400, from about 5000 to about 5200, from about 5200 to about 6000, from about 5200 to about 5800, from about 5200 to about 5600, from about 5200 to about 5400, from about 5400 to about 6000, from about 5400 to about 5800, from about 5400 to about 5600, from about 5600 to about 6000, from about 5600 to about 5800, or from about 5800 to about 6000) genomic intervals. A genomic interval can be any appropriate length. For example, a genomic interval can be the length of an amplicon sequenced as described herein. For example, a genomic interval can be the length of a chromosome arm. In some cases, a genomic interval can include from about 100 to about 125,000,000 (e.g., about 100 to about 100,000,000, about 100 to about 75,000,000, about 100 to about 50,000,000, about 100 to about 25,000,000, about 100 to about 1,000,000, about 100 to about 750,000, about 100 to about 500,000, about 100 to about 250,000, about 100 to about 100,000, about 100 to about 75,000, about 100 to about 50,000, about 100 to about 25,000, about 100 to about 1,000, about 100 to about 500, about 500 to about 125,000,000, about 500 to about 100,000,000, about 500 to about 75,000,000, about 500 to about 50,000,000, about 500 to about 25,000,000, about 500 to about 1,000,000, about 500 to about 750,000, about 500 to about 500,000, about 500 to about 250,000, about 500 to about 100,000, about 500 to about 75,000, about 500 to about 50,000, about 500 to about 25,000, about 500 to about 1,000, about 1,000 to about 125,000,000, about 1,000 to about 100,000,000, about 1,000 to about 75,000,000, about 1,000 to about 50,000,000, about 1,000 to about 25,000,000, about 1,000 to about 1,000,000, about 1,000 to about 750,000, about 1,000 to about 500,000, about 1,000 to about 250,000, about 1,000 to about 100,000, about 1,000 to about 75,000, about 1,000 to about 50,000, about 1,000 to about 25,000, about 25,000 to about 125,000,000, about 25,000 to about 100,000,000, about 25,000 to about 75,000,000, about 25,000 to about 50,000,000, about 25,000 to about 25,000,000, about 25,000 to about 1,000,000, about 25,000 to about 750,000, about 25,000 to about 500,000, about 25,000 to about 250,000, about 25,000 to about 100,000, about 25,000 to about 75,000, about 25,000 to about 50,000, about 50,000 to about 125,000,000, about 50,000 to about 100,000,000, about 50,000 to about 75,000,000, about 50,000 to about 50,000,000, about 50,000 to about 25,000,000, about 50,000 to about 1,000,000, about 50,000 to about 750,000, about 50,000 to about 500,000, about 50,000 to about 250,000, about 50,000 to about 100,000, about 50,000 to about 75,000, about 75,000 to about 125,000,000, about 75,000 to about 100,000,000, about 75,000 to about 75,000,000, about 75,000 to about 50,000,000, about 75,000 to about 25,000,000, about 75,000 to about 1,000,000, about 75,000 to about 750,000, about 75,000 to about 500,000, about 75,000 to about 250,000, about 75,000 to about 100,000, about 100,000 to about 125,000,000, about 100,000 to about 100,000,000, about 100,000 to about 75,000,000, about 100,000 to about 50,000,000, about 100,000 to about 25,000,000, about 100,000 to about 1,000,000, about 100,000 to about 750,000, about 100,000 to about 500,000, about 100,000 to about 250,000, about 250,000 to about 125,000,000, about 250,000 to about 100,000,000, about 250,000 to about 75,000,000, about 250,000 to about 50,000,000, about 250,000 to about 25,000,000, about 250,000 to about 1,000,000, about 250,000 to about 750,000, about 250,000 to about 500,000, about 500,000 to about 125,000,000, about 500,000 to about 100,000,000, about 500,000 to about 75,000,000, about 500,000 to about 50,000,000, about 500,000 to about 25,000,000, about 500,000 to about 1,000,000, about 500,000 to about 750,000, about 750,000 to about 125,000,000, about 750,000 to about 100,000,000, about 750,000 to about 75,000,000, about 750,000 to about 50,000,000, about 750,000 to about 25,000,000, about 750,000 to about 1,000,000, or about 1,000,000 to about 125,000,000) nucleotides. In some embodiments, clusters of genomic intervals are formed using any appropriate method known in the art. In some embodiments, clusters of genomic intervals are formed based on shared amplicon features of the genomic intervals (see, e.g., Douville et al. PNAS 201 115(8):1871-1876, which is herein incorporated by reference in its entirety).

In some embodiments, methods described herein can identify one or more chromosomal abnormalities include assessing a genome (e.g., a genome of a subject) for the presence or absence of one or more chromosomal abnormalities (e.g., aneuploidies, focal amplification, tumor mutation burden, or difference in cfDNA size). The presence or absence of one or more chromosomal anomalies in the genome of a subject can, for example, be determined by sequencing a plurality of amplicons obtained from a biological sample (e.g., a DNA sample) obtained from the subject to obtain sequencing reads, and grouping the sequencing reads into clusters of genomic intervals. In some cases, read counts of genomic intervals can be compared to read counts of other genomic intervals within the same sample. In some cases where read counts of genomic intervals are compared to read counts of other genomic intervals within the same sample, a second (e.g., control or reference) sample is not assayed. In some cases, read counts of genomic intervals can be compared to read counts of genomic intervals in another sample. For example, when using methods described herein to identify genetic relatedness, polymorphisms (e.g., somatic mutations), and/or microsatellite instability, genomic intervals can be compared to read counts of genomic intervals in a reference sample. A reference sample can be a synthetic sample. A reference sample can be from a database. In some cases where methods described herein are used to identify anomalies (e.g., aneuploidies, focal amplification, tumor mutation burden, or difference in cfDNA size), a reference sample can be a normal sample obtained from the same cancer patient (e.g., a sample from the cancer patient that does not harbor cancer cells) or a normal sample from another source (e.g., a patient that does not have cancer). In some cases where method and materials described herein are used to identify abnormalities (e.g., aneuploidies, focal amplification, tumor mutation burden, or difference in ctDNA size), a reference sample can be a normal sample obtained from the same patient.

In some embodiments, methods described herein are used for detecting aneuploidy in a genome of subject. For example, a plurality of amplicons obtained from a sample obtained from a subject can be sequenced, the sequencing reads can be grouped into clusters of genomic intervals, the sums of the distributions of the sequencing reads in each genomic interval can be calculated, a Z-score of a chromosome arm can be calculated, and the presence or absence of an aneuploidy in the genome of the subject can be identified. The distributions of the sequencing reads in each genomic interval can be summed. For example, sums of distributions of the sequencing reads in each genomic interval can be calculated using the equation

$${\sum }_{1}^{I} {R}_{i} ∼ N ( {\sum }_{1}^{I} {μ}_{i} , {\sum }_{1}^{I} {σ}_{i}^{2} ) ,$$

where Rᵢ is the number of sequencing reads, I is the number of clusters on a chromosome arm, Nis a Gaussian distribution with parameters μᵢ and σᵢ², μᵢ is the mean number of sequencing reads in each genomic interval, and σᵢ² is the variance of sequencing reads in each genomic interval. A Z-score of a chromosome arm can be calculated using any appropriate technique. For example, a Z-score of a chromosome arm can be calculated using the quantile function

$$1 - CDF ( {\sum }_{1}^{I} {μ}_{i} , {\sum }_{1}^{I} {σ}_{i}^{2} ) .$$

The presence of an aneuploidy in the genome of the subject can be identified in the genome of the subject when the Z-score is outside a predetermined significance threshold, and the absence of an aneuploidy in the genome of the subject can be identified in the genome of the subject when the Z-score is within a predetermined significance threshold. The predetermined threshold can correspond to the confidence in the test and the acceptable number of false positives. For example, a significance threshold can be ±1.96, ±3, or ±5. In some embodiments, methods and materials described herein employ supervised machine learning. In some embodiments, supervised machine learning can detect small changes in one or more chromosome arms. For example, supervised machine learning can detect changes such as chromosome arm gains or losses that are often present in a disease or disorder associated with chromosomal anomalies, such as cancer or congenital anomalies. In some embodiments, supervised machine learning can detect changes such as chromosome arm gains or losses that are present in a preimplantation embryo (e.g., a preimplantation embryo generated by in vitro fertilization methods). In some cases, supervised machine learning can be used to classify samples according to aneuploidy status. For example, supervised machine learning can be employed to make genome-wide aneuploidy calls. In some cases, a support vector machine model can include obtaining an SVM score. An SVM score can be obtained using any appropriate technique. In some cases, an SVM score can be obtained as described elsewhere (see, e.g., Cortes 1995 Machine learning 20:273-297; and Meyer et al. 2015 R package version:1.6-3). At lower read depths, a sample will typically have a higher raw SVM score. Thus, in some cases, raw SVM probabilities can be corrected based on the read depth of a sample using the equation

$$log ( 1 - \frac{1}{r} ) = Ax + B ,$$

where r is the ratio of the SVM score at a particular read depth/minimum SVM score of a particular sample given sufficient read depth. A and B can be determined as described in Example 1. For example, A=−7.076*10\hat{ }−7, x=the number of unique template molecules for the given sample, and B=−1.946*10\hat{ }−1.

In some embodiments, provided herein are methods that can be used to detect copy number variants (CNVs) of indeterminate length. In some embodiments, provided herein are methods to detect copy number variation of near-fixed length. In some embodiments, detecting copy number variation include calculating the values of one or more variables. In some embodiments, using a log ratio of the observed test sample and WALDO predicted values from every 500 kb interval across each chromosomal arm, a circular binary segmentation algorithm can be applied to determine copy number variants throughout each chromosome arm. For example, copy number variant≤5 Mb in size can be flagged. In some embodiments, the flagged CNVs can be removed before, contemporaneously with, and/or after the analysis. In some embodiments, small CNVs may be used to assess microdeletions or microamplifications. For example, microdelections or microamplifications occur in DiGeorge Syndrome (chromosome 22q11.2 or in breast cancers (chromosome 17q12).

In some embodiments, the method further comprises detecting the chromosomal abnormality in the DNA sample and identifying the chromosomal abnormality as a prognostic biomarker in the subject. In some embodiments, the chromosomal abnormality is selected from aneuploidy, a focal amplification, tumor mutation burden, chromosomal copy number changes, or cfDNA size. In some embodiments, the detection of chromosomal copy number changes determines a type of cancer in the subject.

Examples of chromosomal abnormalities that can be detected using methods described herein include, without limitation, numerical disorders, structural abnormalities, allelic imbalances, and microsatellite instabilities. A chromosomal abnormality can include a numerical disorder. For example, a chromosomal anomaly can include an aneuploidy (e.g., an abnormal number of chromosomes). In some cases, an aneuploidy can include an entire chromosome. In some cases, an aneuploidy can include part of a chromosome (e.g., a chromosome arm gain or a chromosome arm loss). Examples of aneuploidies include, without limitation, monosomy, trisomy, tetrasomy, and pentasomy. A chromosomal anomaly can include a structural abnormality. Examples of structural abnormalities include, without limitation, deletions, duplications, translocations (e.g., reciprocal translocations and Robertsonian translocations), inversions, insertions, rings, and isochromosomes. Chromosomal anomalies can occur on any chromosome pair (e.g., chromosome 1, chromosome 2, chromosome 3, chromosome 4, chromosome 5, chromosome 6, chromosome 7, chromosome 8, chromosome 9, chromosome 10, chromosome 11, chromosome 12, chromosome 13, chromosome 14, chromosome 15, chromosome 16, chromosome 17, chromosome 18, chromosome 19, chromosome 20, chromosome 21, chromosome 22, and/or one of the sex chromosomes (e.g., an X chromosome or a Y chromosome). For example, aneuploidy can occur, without limitation, in chromosome 13 (e.g., trisomy 13), chromosome 16 (e.g., trisomy 16), chromosome 18 (e.g., trisomy 18), chromosome 21 (e.g., trisomy 21), and/or the sex chromosomes (e.g., X chromosome monosomy; sex chromosome trisomy such as XXX, XXY, and XYY; sex chromosome tetrasomy such as XXXX and XXYY; and sex chromosome pentasomy such as XXXXX, XXXXY, and XYYYY). For example, structural abnormalities can occur, without limitation, in chromosome 4 (e.g., partial deletion of the short arm of chromosome 4), chromosome 11 (e.g., a terminal 11q deletion), chromosome 13 (e.g., Robertsonian translocation at chromosome 13), chromosome 14 (e.g., Robertsonian translocation at chromosome 14), chromosome 15 (e.g., Robertsonian translocation at chromosome 15), chromosome 17 (e.g., duplication of the gene encoding peripheral myelin protein 22), chromosome 21 (e.g., Robertsonian translocation at chromosome 21), and chromosome 22 (e.g., Robertsonian translocation at chromosome 22).

### Method of Disease Monitoring in a CNS Patient

Provided herein are methods of disease monitoring in a subject having a central nervous system (CNS) cancer that include (a) obtaining a DNA sample from the subject; (b) amplifying a plurality of chromosomal sequences in the DNA sample with a pair of primers complementary to the plurality of chromosomal sequences to form a plurality of amplicons; (c) determining at least a portion of a nucleic acid sequence of one or more of the plurality of amplicons; (d) mapping the determined nucleic acid sequence to a reference chromosome; (e) dividing the DNA sample into a plurality of genomic intervals; (f) quantifying a plurality of features for the one or more amplicons mapped to the genomic intervals; (g) comparing the plurality of features in a first genomic interval with the plurality of features in one or more different genomic intervals and detecting a chromosomal abnormality in the DNA sample from the subject; and (h) repeating steps (a)-(g) at multiple time points, thereby monitoring progression of the CNS cancer in the subject.

As used herein, “disease monitoring” can refer to an ongoing, timely, and systematic collection and analysis of information of the extent of a disease, screening of test results, disease progression after treatment, and surveillance of survival or death of a subject. During active disease monitoring, specific exams and tests are performed on a regular schedule. In some embodiments, disease monitoring can be used to avoid or delay the need for treatments such as radiation therapy or surgery. In some embodiments, disease monitoring can be used for treatment of the disease (e.g., cancer).

In some embodiments, method described herein can be performed on a regular schedule at multiple time points. In some embodiments, method described herein can be performed daily, every 7 days, every 14 days, every 21 days, every 28 days, every month, every 2 months, every 4 months, every 6 months, or every year. In some embodiments, the multiple time points comprise every week, every two weeks, every four weeks, every six weeks, or every eight weeks. In some embodiments, the repeating step (h) is performed at a time point after an anti-cancer treatment for the CNS cancer is administered to the subject. In some embodiments, the repeating step can be performed 24 hours after, 7 days after, 14 days after, 21 days after, 28 days after, a month after, 2 months after, 4 months after, 6 months after, or a year after the anti-cancer treatment is administered.

In some embodiments, the repeating step (h) further comprises determining minimal residual disease (MRD) in the subject. As used herein, the term “minimal residual disease (MRD)” can refer to the disease that remains in the subject after treatment. In some embodiments, the methods described herein can be used to detect MRD in a subject after an anti-cancer treatment is administered. In some embodiments, the anti-cancer treatment can include chemotherapy, radiation therapy, surgery, or immunotherapy. In some embodiments, the anti-cancer treatment can include ionizing radiation, a chemotherapeutic agent, a therapeutic antibody, a checkpoint inhibitor, or any combination thereof.

### Method of Treatment for CNS Cancer

Provided herein are methods of treating a CNS tumor in a subject in need thereof that includes (a) diagnosing the subject as having the CNS tumor according to any one of the methods described herein; and (b) administering an anti-cancer treatment to the subject.

In some embodiments, methods described herein can be used for identifying and/or treating a disease (e.g., cancer) associated with one or more chromosomal abnormalities (e.g., one or more chromosomal abnormalities identified as described herein, such as, without limitation, an aneuploidy). In some cases, a DNA sample (e.g., a genomic DNA sample) obtained from a subject can be assessed for the presence or absence of one or more chromosomal abnormalities. For example, a subject (e.g., a human) can be identified as having a disease based on the presence of one or more chromosomal anomalies can be treated with one or more cancer treatments. In some embodiments, a subject identified as having cancer based, at least in part, on the presence of one or more chromosomal anomalies is treated with one or more cancer treatments.

In some embodiments, a subject identified as having a disease or disorder associated with one or more chromosomal anomalies as described herein (e.g., based at least in part on the presence of one or more chromosomal anomalies, such as, without limitation, an aneuploidy) can have the disease or disorder diagnosis confirmed using any appropriate method. In some embodiments, a method of identifying a subject as having a disease or disorder (e.g., a central nervous system (CNS) cancer) can include (a) obtaining a DNA sample from the subject; (b) determining one or more chromosomal abnormalities in the DNA sample, thereby identifying the subject as having the disease or disorder by detecting the chromosomal abnormality in the DNA sample from the subject. Examples of methods that can be used to confirm the presence of one or more chromosomal anomalies include, without limitation, karyotyping, fluorescence in situ hybridization (FISH), quantitative PCR of short tandem repeats, quantitative fluorescence PCR (QF-PCR), quantitative PCR dosage analysis, quantitative mass spectrometry of SNPs, comparative genomic hybridization (CGH), whole genome sequencing, and exome sequencing.

In some embodiments, a CNS cancer is a primary brain tumor. In some embodiments, a CNS cancer is a metastatic CNS cancer (e.g., secondary brain tumors). In some embodiments, a CNS cancer can include meningioma, pituitary adenoma, craniopharyngioma, neurofibroma, hemangioblastoma, encephalocele, fibrous dysplasia, glioma, astrocytomas, oligodendrogliomas, glioblastomas, ependymal tumors, hemangiopericytoma, germ cell tumors, chordoma, chondrosarcoma, medulloblastoma, olfactory neuroblastoma, lymphoma, gliosarcoma, rhabdomyosarcoma, paranasal sinus cancer, and atypical teratoid/rhabdoid tumor (AT/RT). In some embodiments, the CNS cancer can include a glioblastoma (GBM), medulloblastoma, parenchymal metastases (PM), leptomeningeal disease (LMD), diffuse large B-cell lymphoma, or CNS lymphoma. In some embodiments, the CNS cancer is glioblastoma (GBM), medulloblastoma, parenchymal metastases (PM), leptomeningeal disease (LMD), diffuse large B-cell lymphoma, or CNS lymphoma.

In some embodiments, the anti-cancer treatment can include chemotherapy, radiation therapy, surgery, or immunotherapy. In some embodiments, the anti-cancer treatment can include ionizing radiation, a chemotherapeutic agent, a therapeutic antibody, a checkpoint inhibitor, or any combination thereof. In some embodiments, the anti-cancer treatment can include a general targeted cancer therapy, wherein the cancer targets can include, but are not limited to, IDH1/2, EGFR, BRCA, BRAF, PIK3CA, KRAS, and HER2-NEU.

### EXAMPLES

The disclosure is further described in the following examples, which do not limit the scope of the disclosure described in the claims.

### Example 1—Repetitive Element Aneuploidy Sequencing System (RealSeqS) in CNS Tumors

### Patient Samples

Patients were recruited as part of an Institutional Review Board-approved, multi-institutional study to develop biomarkers for central nervous system tumors using cerebrospinal fluid. The 4 institutions (Johns Hopkins, University of Michigan, Penn State, The Children Brain Tumor Tissue Consortium (CBTTC)) are tertiary centers that care for patients referred for management of central nervous system tumors. In general, patients underwent sampling on the same day of enrolling and only tumors with radiographic confirmation with contrast enhanced MRI were included in the study. Radiographic findings of disease were based on the findings of a board certified neuroradiologist at each site. In total there were 92 samples in the training set and 190 samples in a validation set. Pathologic diagnosis for all cases was verified by board certified neuropathologists at the site of enrollment. 43 plasma samples were also collected from patients with CNS cancers.

### RealSeqS Conditions

CSF was frozen in its entirety at −80° C. until DNA purification, and the entire volume of CSF (cells plus fluid) was used for DNA purification. The amount of CSF used ranged from 0.5-1 mL. CSF and plasma DNA was purified from healthy individuals and patients with Biochain kit catalog #K5011625MA.

A single primer pair was used to amplify ˜350,000 loci spread throughout the genome. PCR was performed in 25 uL reactions containing 7.25 uL of water, 0.125 uL of each primer, 12.5 uL of NEBNext Ultra II Q5 Master Mix (New England Biolabs cat #M0544S), and 5 μL of DNA. The cycling conditions were: one cycle of 98° C. for 120 s, then 15 cycles of 98° C. for 10 s, 57° C. for 120 s, and 72° C. for 120 s. Each sample was assessed in eight independent reactions, and the amount of DNA per reaction varied from ˜0.1 ng to 0.25 ng. A second round of PCR was then performed to add dual indexes (barcodes) to each PCR product prior to sequencing. The second round of PCR was performed in 25 uL reactions containing 7.25 uL of water, 0.125 uL of each primer, 12.5 uL of NEBNext Ultra II Q5 Master Mix (New England Biolabs cat #M0544S), and 5 μL of DNA containing 5% of the PCR product from the first round. The cycling conditions were: one cycle of 98° C. for 120 s, then 15 cycles of 98° C. for 10 s, 65° C. for 15 s, and 72° C. for 120 s. Amplification products from the second round were purified with AMPure XP beads (Beckman cat # a63880), as per the manufacturer's instructions, prior to sequencing. As noted above, each sample was amplified in eight independent PCRs in the first round. Each of the eight independent PCRs was then re-amplified using index primers in the second PCR round. The sequencing reads from the 8 replicates were summed for the bioinformatic analysis but could also be assessed individually for quality control purposes.

Sequencing was performed on an Illumina HiSeq 4000. The average number of uniquely aligned reads was 10.5 million (interquartile range, 8.0-12.7 million). Any sample with fewer than 2.5M reads was excluded. Depth threshold was the recommended exclusion metric in the initial RealSeqS manuscript.

### Detection of Chromosome Alterations

Fifteen samples from individuals without cancer were used as reference samples; these samples were taken from the training set and not used for the evaluation of performance metrics in the validation set. A separate plasma panel of normals was used for the evaluation of plasma sensitivity. Each experimental sample was then matched to the reference samples that were most similar with respect to the amplicon distributions generated by RealSeqS. The WALDO algorithm compares the normalized read counts of 500-kb intervals to intervals on other chromosome arms in the same sample. Its normalization is “within-sample”. The intervals are aggregated across the entire length of the chromosome arm to produce an arm-level statistical significance (Z_{w}). The 39 nonacrocentric Z_{w} serve as features that are integrated and modeled with a support machine learning from a collection of normal euploid plasma samples and plasma samples from aneuploid cancers. The model generates a Global Aneuploidy Score (GAS) that discriminates between aneuploid and euploid samples. No samples in the GAS training set overlap with samples in this study.

### Detection of Focal Amplifications

A series for focal changes were considered during training set evaluation. To identify focal amplifications, we first identified genomic coordinates from the University of California Santa Cruz genome browser. RealSeqS amplicons overlapping with the gene of interest and an additional±100 amplicons (˜1 MB) flanking the gene were identified. For each sample: the read count across these amplicons was determined. Statistical significance for each gene was calculated (Eq 1). The λ was calculated from the CSF non-cancer samples in the training set for use in the CSF samples and re-calculated from the panel of plasma normals for use in the plasma samples.

$${Z}_{gene} = \frac{{Observed}_{gene} - {λ}_{gene}}{\sqrt{{λ}_{gene}}} \qquad [ Equation 1 ]$$

### Detection of Somatic Mutations

Adapters were trimmed using cutadapt2 and aligned to the genome using Bowtie2. Somatic mutations were identified using Mutect2 default parameters. Multi-allelic, poor mapping quality, poor base-quality, and clustered variants were discarded. Only autosomal chromosomes were considered and a hard allele frequency cutoff of <0.35 was used to discard germline variants. Then the remaining single nucleotide changes were counted. Runs with low quality are more likely to have an increased number of variants due to sequencing errors. No samples from runs with Q30<75 (all cycles) were used for mutation analysis. Numerous studies have demonstrated that low concentration during PCR increases the number of artifactual somatic mutations. All samples were quantified with qPCR and a cutoff of <0.03 ng/uL was selected and were not used for mutation analysis.

### Patient Characteristics

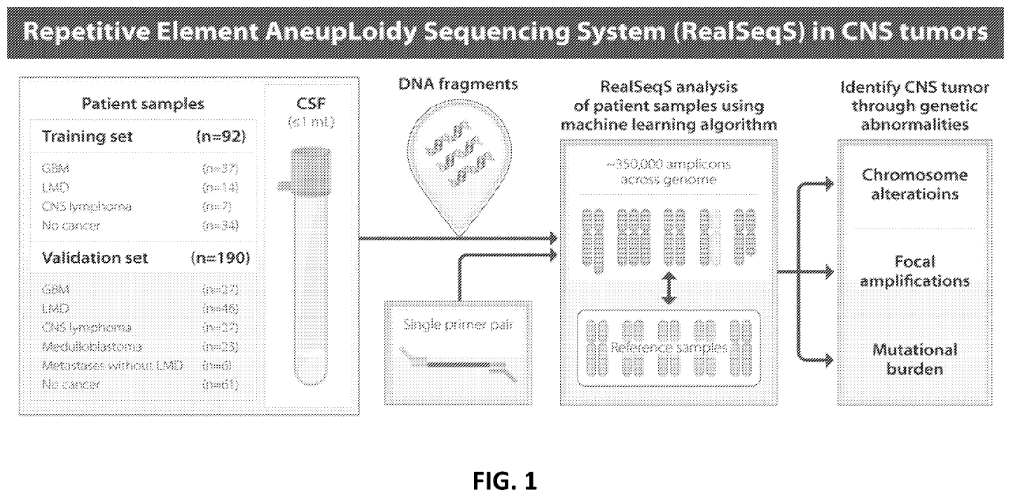

The cohort consisted of 92 patients in the training set, comprising 37 samples from patients with GBM, 14 with leptomeningeal disease, 7 with CNS lymphoma, and 34 without cancer, and 190 in the validation set, consisting of 27 samples from patients with GBM, 46 with leptomeningeal disease, 27 with lymphoma, 23 with medulloblastoma, 6 metastases without leptomeningeal disease (FIG. 1), and 61 without cancer. Medulloblastoma and metastases without leptomeningeal disease samples were not included in the training set. Samples were pre-specified into training and validation cohorts based on the sample source and the time in which they were completed. A subset of samples from Johns Hopkins were initially completed and labeled as training samples. To reduce potential cohort biases and machine learning overfitting, all samples from the Penn State University, Children's Hospital of Philadelphia, and the University of Michigan were labeled as validation samples. The remaining Johns Hopkins samples not completed in the initial batch of samples were included into the validation cohort. Of 43 plasma samples, 32 were from patients with GBM and 11 were from patients with medulloblastoma.

### Training Set

The goal of this example was to discriminate CSF from patients with central nervous tumors from those without cancers. Sensitivity was determined by the fraction of patients with cancer above a given threshold while specificity was determined as the fraction of patients without cancer less than this threshold. The training set is used to examine the utility of 3 possible approaches: global aneuploidy, focal amplifications, and somatic mutation burden. Upon examination of each approach, the optimal threshold to separate cancer and non-cancers is determined for use in the validation set.

First, Z_{w} scores for each of the 39 nonacrocentric chromosome arms in each sample were calculated. These chromosome arm-level Zw scores were then integrated in a single GAS. The GAS reflects the likelihood a sample of interest contains the presence aneuploidy. Supervised machine-learning has improved performance over naïve statistical approaches in lower tumor admixtures by more effectively modeling technical noise, NGS artifacts, and cancer aneuploidies. For clinical applications, it was considered that the medical consequence of missing a tumor (false negative) was higher than the cost of false positives. Since no non-cancer samples had an evaluated GAS. A value of >0.25 was selected as the threshold to be considered aneuploid. Given limited number of non-cancer samples in the training set, a specificity of >95% was targeted.

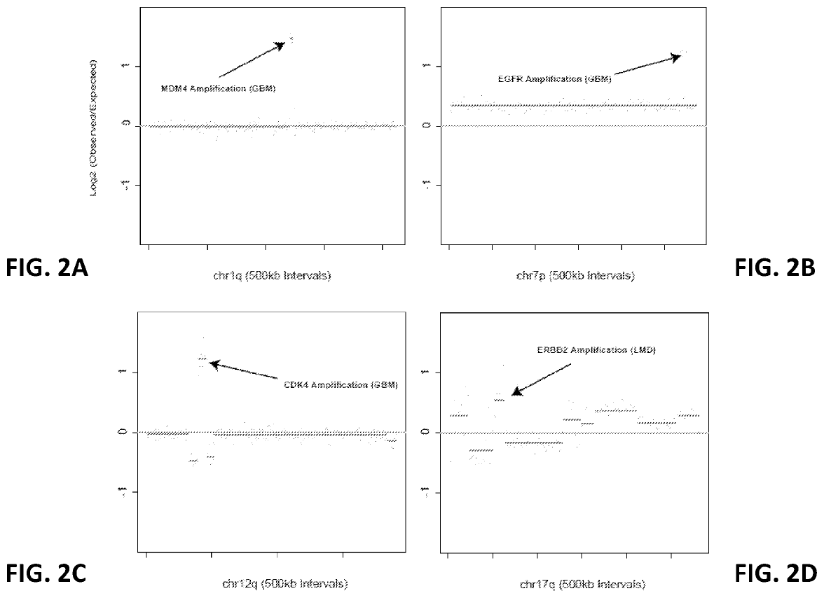

The GAS correctly identified 56.9% of CNS cancers (51.3% GBM, 85.7% LMD, and 28.6% LYM) in the training set, where it is believed this represents an underestimate of aneuploidy in these samples. The GAS was pre-built using a training set of cancers of multiple origins. There are not enough CNS cancers in the training set to reliable re-build a score specifically tailored for CNS tumors. To design a CNS specific aneuploidy caller without re-building a new ML model from the limited number of training examples, a set of candidate CNS focal amplifications were designed based on CNS cancer in The Cancer Genome Atlas (TCGA). The CNS focal panel consists of MDM4, EGFR, CDK4, HER2, c-MYC, MYD88, and CD79B. For each gene, Z_{gene} was calculated. Various threshold for positivity was considered and a cutoff of >10 was selected. 4 representative cancers are illustrated with focal amplifications in FIGS. 2A-2D. In the training set, no non-cancer had focal changes. Upon reviewing the candidate set of genes, c-MYC, MYD88, and CD79B did not identify any additional samples compared to the GAS and were dropped from the candidate list for the validation set. The focal panel detected 27.6% of CNS cancers (24.3% GBM, 42.9% LMD, 14.3% LYM) and 8% of cancers missed with GAS.

Numerous studies have used the number of somatic mutations (Tumor Mutation Burden) to indicate the presence of cancer. Whole genome sequencing, exome sequencing, or gene panels can identify somatic mutations with functional consequences. None of the mutations found in RealSeqS, however, have functional consequences because the loci do not fall in the coding region of the genome. It was previously reported that the number of mutations in repetitive elements is proportional to the number of mutations in exome sequencing. Given the strong correlation, it was hypothesized that large numbers of somatic mutations in RealSeqS could reliably detect samples that were missed with aneuploidy. >39 was selected based on the highest non-cancer in the training set to indicate positivity. This cutoff identified 25.9% cancers (35.1% GBM, 7.1% LMD, 14.3% lymphomas) and a sensitivity of 20% in samples missed with aneuploidy. It was believed the reason behind LMD had low sensitivity was not biological but technical. The LMD training samples had a depth of 5.7M with 5 of 14 samples had fewer than 5M reads. GBM had a median depth of 9.3M and no samples<5M; LYM a median depth of 13.3M with no samples<5M; and the non-cancers a median depth of 7.7M and no samples<5M. It is not surprising the statistical power to detect cancer through TMB is proportional to the depth of sequencing used. Finally, all three approaches was integrated using an OR gate which detected 69.0% cancers (67.6% GBM, 85.7% LMD, 42.9% lymphomas) and correctly labeling all non-cancers.

### Validation Set

The validation set provided an opportunity to independently assess the sensitivity and specificity of RealSeqS in CSF. Note the validation set included samples from 3 outside institutions from the training set. Many multi-institution biomarker manuscripts report dramatically lower performance when samples are taken from different institutional cohorts. To ensure a truly unbiased representation of performance, it was made sure that the validation samples included samples from institutions not represented in the training set.

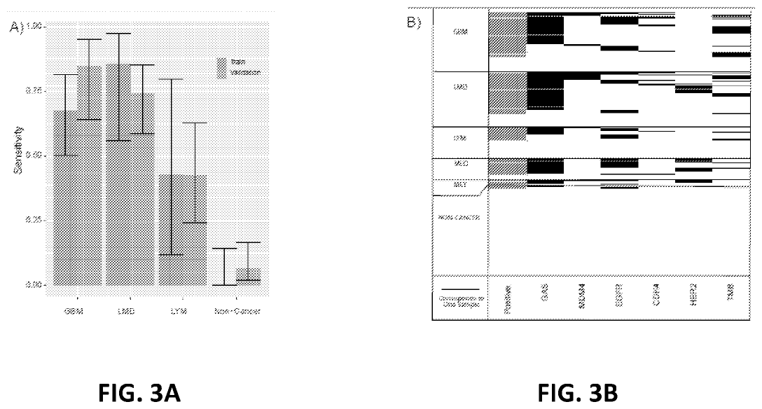

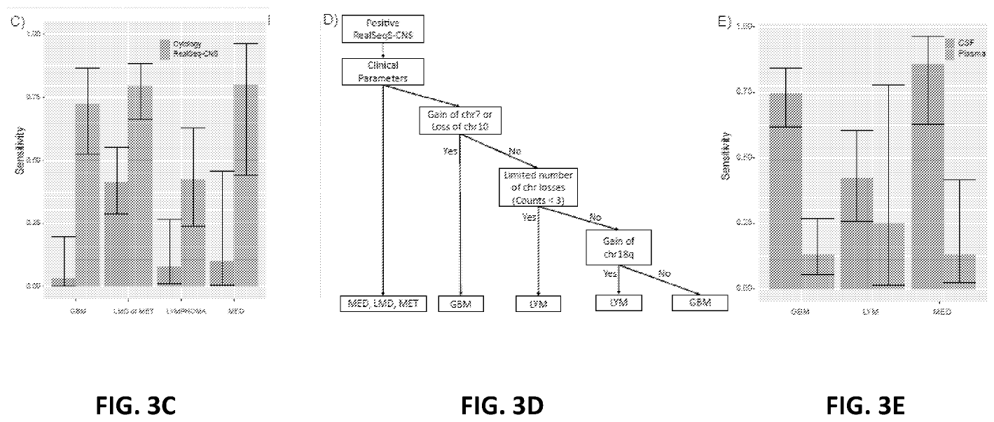

The RealSeqS-CNS algorithm was applied with the pre-defined thresholds from above to the validation samples. RealSeqS-CNS detected 71.3% cancers (85.1% GBM, 73.9% LMD, 44.4% LYM, 78.2% medulloblastoma, and 83.3% metastasis) with a specificity of 93.4% in the non-cancers (FIGS. 3A-3B)

Of the positive validation cancers, 55.0% were detected by the GAS, 49.6% by the focal panel, and 14.7% with TMB. Of the positive cancers, 10.9% were detected by all three metrics; 56.5% by at least 2; and 43.5% by only one. Among the false positives, 2 of 4 were GAS false positives and the other 2 were focal panel false positives. None of the false positives had more than one metric positive.

No sample diagnosis, (GBM, LMD, LYM, non-cancer), present in both the training and validation sets had statistically different detection rates (P>0.05 Two Proportion Z-Test). Samples with LMD had lower sensitivity than samples from with metastatic cancers without leptomeningeal disease (73.9% LMD vs 83.3% MET). It was believed this could have been due to the smaller number of samples (n=46 LMD vs n=6 MET). Given a larger cohort of samples with metastatic disease without leptomeningeal disease a lower detection rate than LMD was anticipated.

### Comparison to Pathology

Pathology review of CSF is standard of care for several CNS neoplasms, but sensitivity remains low typically <50%. Frequently CSF pathology remains inconclusive necessitating a surgical biopsy. For cases with matched pathology, RealSeqS-CNS and pathology sensitivity was compared. RealSeqS-CNS is more sensitive for all cancers⁷0.0% vs 23% (FIG. 3C). In cases with positive pathology, RealSeqS-CNS detects 82% of cancer but sensitivity increases even further-detecting 66.3% of CNS cancers when pathology is indeterminate or negative.

Expanded Analysis of Chromosome Arm Aneuploidies One classifier was designed based on the training set so that the validation set could be rigorously evaluated. Upon completion of the naïve assessment of the Validation Set, it was thought to be of interest to full characterize aneuploidy specific to each CNS cancers. To explore this question, the training and validation samples were both combined.

First, the degree of aneuploidy in samples was assessed using the total number of arms gained or lost (z>3 or z<−3). LMD had the highest degree of aneuploidy with a mean of 17 arms altered followed by MET with 15 arms altered, MED with 13 arms, GBM with 10 arms and LYM with the smallest degree of aneuploidy with 6 arms. The degree is of aneuploidy is in line with previous CNS aneuploidy studies. Later stage cancers such as LMD and MET have a higher degree of aneuploidy than GBM, LYM, and MED. The degree of aneuploidy in LYM is lower than GBM.

Next, it was asked whether the representation of specific aneuploidies could adequately distinguish CNS cancer types. Given the wide range of cancer types in the study, the investigation was limited to only positive GBM and LYM samples. MED is a childhood cancer—age alone is sufficient to differentially exclude from CNS type prediction. LMD and MET are both late-stage malignancies representing a sufficiently distinct clinical workup before CSF sampling. GBM and LYM, however, are radiographically very similar but face very drastically different clinical approaches and outcomes depending on diagnosis.

Based on a review of aneuploidy in tumors from TCGA, LYM and GBM both have a high degree of homogeneity in the representation of arm level events for their respective cancers types. GBM frequently has a gain on 7p and 7q and losses on 10p and 10q—all infrequently observed in LYM. Conversely, LYM often has a gain on 18q and few chromosome arm losses. A simple decision tree was generated (FIG. 3D) using specific aneuploidies in the TCGA to discriminate positive GBM and LYM cancers. When developing the tree, the tradeoff is weighed between under and over calling GBM and LYM as well as the overall positivity rate. Given the severity of GBM as well the lower overall survival rate, it was prioritized calling GBM over LYM when there was uncertainty in the representation of aneuploidies progressing down the decision tree. 73.0% of GBM and LYM cancers (79.2% of GBM and 53.3% of LYM) were accurately predicted.

Analysis of Plasma from Patients with CNS Cancers Given that venipuncture is more accessible than CSF sampling it was asked how sensitivity of tumor DNA detection in plasma compares to that in CSF. 65 plasma samples were scored from CNS cancer patients and a set of 185 previously published non-cancer plasmas using the RealSeqS-CNS approach. The same pre-defined thresholds were applied. The GAS (>0.25) detected 13% of GBM, 25% LYM, and 13% of MED while only miscalling 1.1% of the non-cancer controls. No cancers were detected using the CNS focal panel. The same 2 GAS false positives were miscalled, and no new false positives were identified. The somatic mutation count, however, could not distinguish cancers and non-cancers in plasma. The cutoff of >39 somatic mutations identified 57.8% of the non-cancers and 67.7% of the CNS cancer plasmas. The higher somatic mutation background rate may be explained by age related clonal hematopoiesis. In the non-cancer cohort, individuals>65 years old had an average somatic mutation count of 67.1 while individuals<30 years old had an average of 39.9. Dropping the somatic mutation count the RealSeqS-CNS approach has a sensitivity of 13.8% and specificity of 98.9% (FIG. 3E).

Of the 65 plasmas plasma samples there were matching CSF for 35 samples. In the matched samples, 65.7% of the CSF samples were positive while only 22.9% of the plasmas were positive. Even though the matched plasma had notably lower sensitivity, 3 additional cases were detected in plasma improving the overall sensitivity of a standalone CSF test from a sensitivity of 65.7% to 74.3%.

### CSF DNA Size

Although the primary aims of the study estimate RealSeqS-CNS performance and characterize aneuploidy, other possible biomarkers were investigated in the cohort of CNS cancers. Cell-free DNA (cfDNA) size has been extensively studied and was one of the earliest cancer biomarkers reported in blood across multiple cancer types. DNA in CSF consists of both cell free DNA (cfDNA) and genomic DNA from cells but the size and relative contribution of each, however, has not been well characterized in CNS cancers. Here, it was investigated whether size as evaluated with RealSeqS can discriminate between CNS cancers and non-cancers in CSF. RealSeqS consists of ˜350,000 amplicons with sizes ranging from 70-500 base pairs (bps) with most amplicons ranging from 80-85 bps. cfDNA consists of small fragments typically 160-180 bps and will predominantly amplify smaller loci. Genomic DNA, on the other, is not size limited and can amplify loci of all sizes. By calculating the relative abundance of each size after normalizing for the total number of loci at each size, the empirical probability mass function (ePMF) can be estimated. The proportion of DNA from cfDNA was determined as the relative contribution to loci<200 bps.

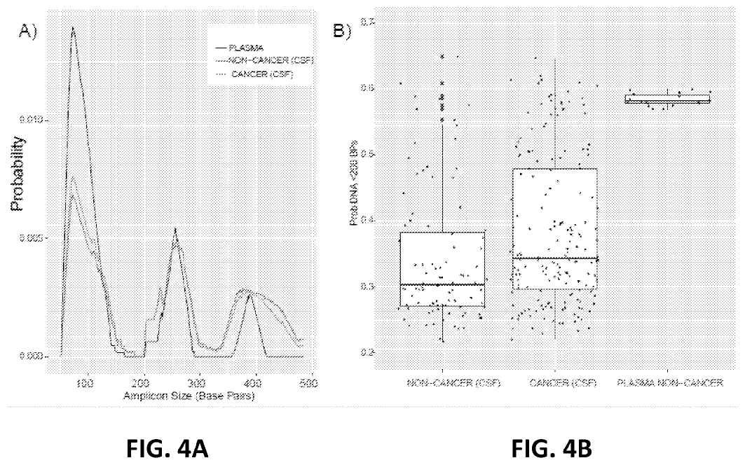

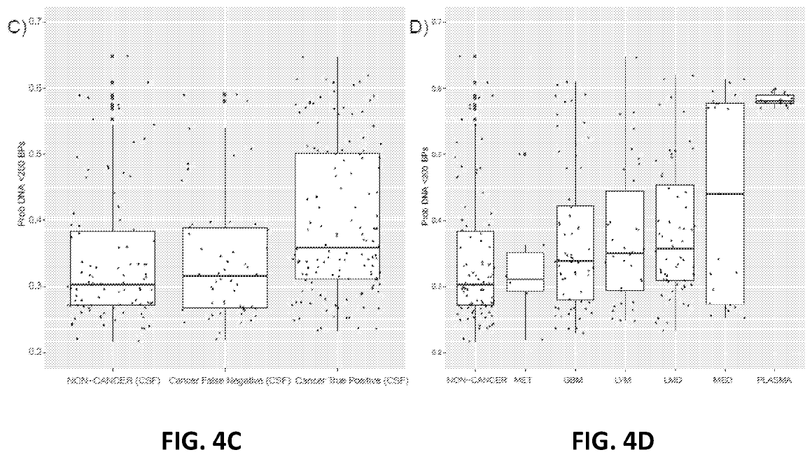

These distributions were illustrated across non-cancer plasma samples, CSF from cancer samples, and CSF from non-cancers (FIG. 4A). An increase in smaller loci was seen in cancer samples compared to non-cancer samples representing an 11.2% increase in cfDNA (p<0.002 one-sided t-test) (FIG. 4B). The relative proportion of cfDNA when only considering cancers with positive RealSeqS-CNS calls show greater separation with a 16.8% increase (p<0.0002 one-sided t-test) in cfDNA compared to non-cancers (FIG. 4C). When comparing individual cancer types, it was seen that MED has the largest proportion of its DNA from cfDNA followed by LMD, LYM, GBM, and finally MET (FIG. 4D). The wide variability and limited number of MET (n=6) samples may account for the smallest difference from the non-cancer controls which was unexpected. Even though the average cancer sample exhibits an increase in cfDNA, wide variability was observed across all samples and the size metric alone would not be sufficient to predict cancer status.

### Example 2—Repetitive Element AneupLoidy Sequencing in CSF (Real-CSF)

### Patient Characteristics

Two independent cohorts of patients were evaluated in this study: a training set and a validation set. The training set was composed of CSF samples from 85 patients, 31 with GBM, 13 with metastasis from primary tumors outside the brain, 7 with lymphoma, and 34 without cancer. The validation set was composed of CSF samples from 195 patients, 27 with GBM (five of which were pediatric H3K27M diffuse midline gliomas), 52 with metastasis from primary tumors outside the brain, 27 with CNS lymphoma, 23 with medulloblastoma, and 62 without cancer (FIG. 1). Thirteen metastatic samples were previously analyzed and reported. The CSF was obtained in almost all cases from lumbar puncture or aspiration from a ventricular catheter placed as part of standard of care.

### Rationale and Background of the Assay

Central nervous system (CNS) neoplasms comprise a heterogenous class of tumors and an equally diverse landscape of genetic alterations. Identifying the optimal combination of genetic markers that could encompass all CNS cancers is difficult. There is often insufficient starting material in CSF to query all somatic mutation and translocation across all potential driver genes. Aneuploidy or the presence of an abnormal number of chromosomes is a feature of most CNS cancer cells. Nearly all GBM, medulloblastoma, and metastatic cancers are aneuploid. CNS lymphoma has a notably lower rate of aneuploidy but still occurs in the majority of these cancers (71%)²³. It was hypothesized that aneuploidy could act as a viable biomarker for CNS cancers, with variation in performance based on prevalence of copy number changes.

Here, aneuploidy was evaluated as a potential biomarker with a simple PCR assay that uses a single primer pair to amplify ˜350,000 short interspersed nuclear elements (SINEs) throughout the genome. The PCR products can then be assessed by massively parallel sequencing to identify chromosomal gains and losses as well as focal amplifications and deletions. The efficiency of PCR copying DNA is high (>90%) and even been able to reliably detect aneuploidy in as little as a few pg of DNA—representing half of a diploid cell. Given the limited starting material in CSF, this assay is well suited to evaluate aneuploidy as a possible CNS biomarker. This approach was named Repetitive Element AneupLoidy Sequencing in CSF (Real-CSF).

### Training Set Data

The training set was used to optimize the machine learning algorithms and other aspects of the analytic workflow. It was first assessed whether the presence of large-scale chromosome arm gains and losses (aneuploidy) could detect cancerous lesions with high specificity. To assess the degree of aneuploidy, Z_{w} scores for each of the 39 non-acrocentric chromosome arms in each sample were calculated. These chromosome arm-level Z_{w} scores were then integrated into a single score, called the Global Aneuploidy Score. The Global Aneuploidy Score reflects the likelihood that a sample has gained or lost at least one chromosome, with the magnitude of the score reflecting both the number of chromosome arms that were altered as well as the fraction of cells in the CSF in which these changes occurred.

Based on cross-validation in the training set, a Global Aneuploidy Score threshold of 0.25 was established for subsequent validation. This threshold correctly identified 63% (95% CI 48% to 75%) of the 85 CSF samples from cancer patients—58% of patients with GBM, 92% of patients with metastases to the brain, and 29% of patients with lymphomas. Of the 34 patients with brain lesions but without cancer, none had Global Aneuploidy Scores<0.25, yielding a specificity of 100% (95% CI 90% to 100%)

It was next sought to determine whether the evaluation of focal amplifications of oncogenes, i.e., those involving only a small region surrounding an oncogene rather than the entire chromosome arm on which the oncogene is located, could detect other CNS cancers using data generated with RealSeqS. For this analysis, oncogenes that were relatively frequently amplified in CNS cancers were first selected based on data from The Cancer Genome Atlas (TCGA). Using the training cohort to assess the potential value of these genes, the list was narrowed to four genes—MDM4, EGFR, CDK4, and HER2.

For each of these four genes, a Focal Amplification Score and a threshold for positivity was calculated in an analogous way to that described above for the Global Aneuploidy Score. It was found that 31% (95% CI 20% to 46%) of the 85 CSF samples from patients with CNS cancers scored positively (examples in FIGS. 2A-2D). Using a Boolean OR gate, a sample was defined as positive in Real-CSF if it scored positively either for Global Aneuploidy or a Focal Amplification of any of the four genes. Two thirds (67%, 95% CI 52% to 79%) of the samples from patients with cancers scored positively in this composite Real-CSF assay, including 65% of the patients with GBM, 92% of the patients with metastatic lesions to the brain, 29% of the patients with lymphomas, and no patient without a CNS cancer.

### Validation Set

The validation set provided an opportunity to independently assess the sensitivity and specificity of Real-CSF. Importantly, the validation set included samples from four different institutions, while samples in the training set were all from only one of these four institutions. This multi-institutional acquisition was intentionally designed to minimize confounders that can be observed when a classification method based on samples from a single institution is applied to samples from other institutions. The validation set also included patients with medulloblastoma, a tumor type not represented in the Training Set but expected to exhibit aneuploidy as well as focal amplifications.

Using the thresholds pre-defined by the training set data, 68% of the patients with cancer scored positively (95% CI 59 to 76%). These included 74% of patients with GBM, 73% of patients with metastatic lesions, 41% of patients with lymphomas, and 78% of medulloblastomas. Of the 62 samples from patients without CNS cancers in the validation set, four (6.4%, CI 5.6% to 12%) scored positively in Real-CSF. No sample type present in both the training and validation sets had statistically different detection rates (P>0.05 Two Proportion Z-Test).

### Survival Analysis

There were sufficient follow-up data to analyze progression free and overall survival in subjects with GBM treated at one of the institutions, JHU. Of the 14 newly diagnosed GBM patients, 10 had detectable levels of CSF-tDNA, while 4 did not. The individuals with detectable levels of CSF-tDNA had an odds ratio of 5.1 (p=0.02, log rank test, Figure SlA) for disease progression when compared to those without CSF-tDNA detection. Of the 29 newly diagnosed and recurrent GBM patients, 20 had detectable levels of CSF-tDNA and 9 had undetectable levels. The cases with detectable CSF-tDNA had an odds ratio of 2.4 for poorer overall survival (p=0.011, log rank test).

Concordance with Whole Genome Sequencing

To orthogonally validate the copy number alterations identified by Real-CSF, conventional whole genome sequencing (WGS) was performed on the CSF DNA from 43 patients with CNS cancers and 28 without cancer. The sequencing depth averaged ˜34.3M read pairs and copy number alterations were identified with WisecondorX. Among the 43 cancer samples, Real-CSF identified 106 chromosome arm level gains (z>7.5) and 126 losses (z<−7.5). Nearly all of these gains (96%) and losses (90%) were identified with WGS. The majority of the chromosome arms gains or losses (9 of 17) that were identified with Real-CSF but not with WGS had z-scores (z>5 or z<−5) just below the z-score of [7.5] required for positivity. Of the 28 CSF DNA samples from patients without cancer, 1091 of 1092 of the chromosome arms evaluated (39 arms×28 patients) were identified as euploid by WGS. The one arm that was aneuploid in one patient was chrom 19p, which has been reported to have a relatively high false positive rate with WGS. Notably, Real-CSF, scored all 1092 chromosome arms as euploid.

Comparison with Cytology

Of the 121 cancer patients from either the Training or Validation sets in whom cytology was available, only 28 (23%, 95% CI 16% to 32%) were detectable by cytology. The sensitivity of Real-CSF in the same 121 patients was 69%, considerably higher than that of cytology (FIG. 3C, p<2.2e-16 Binomial Proportions Test). However, not all patients who had positive cytology also scored positively with Real-CSF, or vice versa. Together, either Real-CSF or cytology was positive in 73% (95% CI 64% to 80%) of cases).

Analysis of Plasma from Patients with CNS Cancers

Given that plasma is much more easily accessible than CSF, it was of interest to determine whether plasma could substitute for CSF in RealSeqS assays for aneuploidy or focal amplifications. Methods to compute Global Aneuploidy and Focal Amplification scores in cell free DNA (cfDNA) from plasma from normal individuals and in patients with cancers of organs other than the brain were previously described. In the current study, plasma in 65 patients with CNS cancers were evaluated (GBM, lymphoma, or medulloblastoma). Also, plasma samples from 185 non-cancer individuals (trigeminal neuralgia, hydrocephalus, and neurodegenerative diseases) were evaluated to assess specificity. Positive Global Aneuploidy Scores were obtained in nine of the 65 cancer patients (sensitivity of 14%; 95% CI 6.9% to 25%) and in two of the 185 controls (specificity of 98.9%, 95% CI 96% to 100%). No focal amplifications were observed in the plasma of patients with or without cancer.

Thirty-five of the 65 brain cancer patients who donated plasma had also donated CSF. In these matched samples, 66% (95% CI 48% to 81%) of the CSF samples scored as positive while 23% (95% CI 10% to 40%) of the plasma samples scored as positive. Five patients scored positively in both plasma and CSF. Eighteen of the 35 patients scored positively in CSF but not in plasma, and conversely, three patients scored positively in plasma but not CSF. Thus, at similar specificities, CSF DNA was a more sensitive analyte than plasma cfDNA for the detection of chromosomal alterations (P<0.00001, Z Score for 2 Population Proportions).

### DNA Purification

CSF was frozen in its entirety at −80° C. until DNA purification, and the entire volume of CSF (cells plus fluid) was used for DNA purification. The amount of CSF used for purification ranged from 0.5 to 1 mL. CSF using Biochain reagents according to the manufacturer's instructions (catalog #K5011625MA).

### Real-CSF

A single primer pair was used to amplify ˜350,000 short interspersed nuclear elements (SINEs) spread throughout the genome. PCR was performed in 25 uL reactions containing 7.25 uL of water, 0.125 uL of each primer, 12.5 uL of NEBNext Ultra II Q5 Master Mix (New England Biolabs cat #M0544S), and 5 μL of DNA. The cycling conditions were: one cycle of 98° C. for 120 s, then 15 cycles of 98° C. for 10 s, 57° C. for 120 s, and 72° C. for 120 s. Each sample was assessed in eight independent reactions, and the amount of DNA per reaction varied from −0.1 ng to 0.25 ng. A second round of PCR was then performed to add dual indexes (barcodes) to each PCR product prior to sequencing. The second round of PCR was performed in 25 uL reactions containing 7.25 uL of water, 0.125 uL of each primer, 12.5 uL of NEBNext Ultra II Q5 Master Mix (New England Biolabs cat #M0544S), and 5 μL of DNA containing 5% of the PCR product from the first round. The cycling conditions were: one cycle of 98° C. for 120 s, then 15 cycles of 98° C. for 10 s, 65° C. for 15 s, and 72° C. for 120 s. Amplification products from the second round were purified with AMPure XP beads (Beckman cat # a63880), as per the manufacturer's instructions, prior to sequencing.

Sequencing was performed on an Illumina HiSeq 4000. The sequencing reads from the 8 replicates of each sample were summed for bioinformatic analysis. The average number of the summed, uniquely aligned reads was 10.5 million (interquartile range, 8.0-12.7 million).

### Chromosome Copy Number Alterations in CSF DNA

The copy number alterations for CSF samples were calculated using the following protocol: Generate a reference panel:
1. Select 15 non-cancer CSF samples.
2. Aggregate and sum the read depth into 5,344 non-overlapping autosomal 500-kb intervals.
3. Normalize reads to account for coverage differences.
4. Perform PCA Normalization for the euploid reference panel. This type of normalization is an attempt to mitigate the impact of highly correlated regions. To perform this normalization, we employed the following steps:
   Normalization Training: For all controls (n=C)
1) Bin read counts for each control sample into 5,344 autosomal intervals of 500 kb each.
2) Normalize reads to account for coverage differences.
3) Project the 5,344 500 kb intervals into PCA space.
4) Define a correction factor variable.

$$Correction Factor for 500 kb {Interval}_{1} = \frac{{μ}_{i} {Interval}_{i}}{{Observed}_{i}}$$

5) Calculate the correction factor for each control. Store the correction factor as a 1×C vector.
6) Define a regression model using the following equation.

$$Correction Factor for 500 kb {Interval}_{1} = {β}_{oi} + {β}_{1 i} * {PCA}_{1} + {β}_{2 i} * {PCA}_{2} + {β}_{3 i} * {PCA}_{3} + {β}_{4 i} * {PCA}_{4} + {β}_{5 i} * {PCA}_{5}$$

7) Estimate the β parameters using regression.
8) Store the β parameters for Interval i.
9) Repeat Steps 4-8 for the remaining 5,343 intervals.
   Perform Analysis on a test sample:
1. Aggregate and sum the read depth into 5,344 non-overlapping autosomal 500-kb intervals.
2. Normalization of the test sample:
   1) Bin read counts for a new test sample into 5,344 500 kb intervals.
   2) Normalize reads to account for coverage differences.
   3) Project the test sample into PCA space.
   4) Estimate the correction factor for the test sample on Interval 1.
   5) Normalize the read count for the test sample on Interval 1 by multiplying the observed read count by the estimated correction factor of Interval 1.
   6) Repeat Steps 4 and 5 for the remaining 5343 intervals.
3. Segment the chromosome arm using the circular binary segmentation algorithm (CBS).
5. Aggregate the 500-kb intervals across the chromosome arm and calculate the statistical significance across the length of the chromosome arm (Z_{w}).
6. Repeat this protocol for all chromosome arms.
7. Evaluate the test sample's 39 chromosome arms using a previously built supervised machine learning algorithm. This model generates a Global Aneuploidy Score (GAS) to discriminate between aneuploid and euploid samples. The predictive features of the model are the 39 chromosome arms (Z_{w}). The training examples were 3,999 previously published plasma samples. The negative class of 1348 presumably euploid samples were taken from individuals without cancer. The positive class was taken from 2651 aneuploid samples across 8 different cancer types. A support vector machine (SVM) was specifically built and trained the model with the e1071 package in R, using a radial basis kernel and default parameters.
8. Score the test sample using the supervised-machine learning model from Step 7.
   Chromosome Copy Number Alterations in Plasma cfDNA

To identify copy number alterations in plasma the steps from above were repeated but made one key change. The euploid reference panel was reconstructed using a set of 1,500 euploid plasma samples. The step-by-step protocol was then repeated as above to calculate the statistical significances for each arm and generate Global Aneuploidy Scores.

### Focal Amplifications

RealSeqS amplicons overlapping the genomic coordinates of the gene of interest, plus 1 Mb on either side of the gene, were identified. The summed read counts (Observed_{gene}) across these amplicons were then determined for each sample.

The protocol to calculate the Z score for each gene was calculated in the following way: For the euploid reference panel:
1. For all samples in the reference panel, normalize each locus by dividing by the total autosomal sequencing depth. This enables samples with varying amounts of coverage to be directly comparable.
2. Aggregate the read depth across the gene of interest and surrounding 1 Mb for each sample.
3. Estimate the average read depth across the euploid reference panel (μ_{gene}).
   For each test sample:
4. Calculate the total autosomal sequencing depth (Coverage)
5. Multiply (μ_{gene}) by the observed coverage to estimate the expected number of reads across the gene of interest (λ_{gene}) given the coverage. It was assumed that the count data followed a Poisson distribution.
6. Aggregate the read depth across the gene of interest (Observed_{gene})
7. Calculate the statistical significance

$${Z}_{gene} = \frac{{Observed}_{gene} - {λ}_{gene}}{\sqrt{{λ}_{gene}}}$$

This protocol was followed for both CSF and plasma samples. The only difference between CSF and plasma was the euploid reference panel used to generate the expected depth for each gene, as noted above.

### WGS

WGS on CSF DNA was prepared, wherein an average of 34.3 M unique reads pairs per sample (IQR 29.2M to 38.8M were obtained. Copy number alterations were identified with WisecondorX using 500 kb intervals and default parameters.

### Quantification and Statistical Analysis

Performance comparisons between the training and validation sets were assessed with the Z Score for 2 Population Proportions. The survival statistics were assessed using the log rank test.

What is claimed is:

1. A method of identifying a subject as having a central nervous system (CNS) cancer, the method comprising:
(a) obtaining a DNA sample from the subject;
(b) analyzing a plurality of chromosomal sequences in the DNA sample;
(c) determining at least a portion of a nucleic acid sequence of one or more of the plurality of chromosomal sequences;
(d) mapping the determined nucleic acid sequence to a reference chromosome;
(e) dividing the DNA sample into a plurality of genomic intervals;
(f) quantifying a plurality of features for the one or more nucleic acid sequences mapped to the genomic intervals; and
(g) comparing the plurality of features in a first genomic interval with the plurality of features in one or more different genomic intervals and detecting a chromosomal abnormality in the DNA sample, thereby identifying the subject as having the CNS cancer.

2. The method of claim 1, wherein the subject is not known to have a CNS cancer.

3. A method of monitoring a central nervous system (CNS) cancer in a subject, the method comprising:
(a) obtaining a DNA sample from the subject;
(b) analyzing a plurality of chromosomal sequences in the DNA sample;
(c) determining at least a portion of a nucleic acid sequence of one or more of the plurality of chromosomal sequences;
(d) mapping the determined nucleic acid sequence to a reference chromosome;
(e) dividing the DNA sample into a plurality of genomic intervals;
(f) quantifying a plurality of features for the one or more nucleic acid sequences mapped to the genomic intervals;
(g) comparing the plurality of features in a first genomic interval with the plurality of features in one or more different genomic intervals and detecting a chromosomal abnormality in the DNA sample from the subject; and
(h) repeating steps (a)-(g) at multiple time points, thereby monitoring progression of the CNS cancer in the subject.

4. The method of any one of claims 1-3, wherein the analyzing step (b) comprises amplifying the plurality of chromosomal sequences in the DNA sample with a pair of primers complementary to the plurality sequences to form a plurality of amplicons.

5. The method of any one of claims 1-4, wherein the method further comprises detecting the chromosomal abnormality in the DNA sample and identifying the chromosomal abnormality as a prognostic biomarker in the subject.

6. The method of claim 5, wherein the chromosomal abnormality is selected from aneuploidy, a focal amplification, tumor mutation burden, chromosomal copy number changes, or cfDNA size.

7. The method of claim 6, wherein the detection of chromosomal copy number changes is used to determine a type of cancer in the subject.

8. The method of claim 3, wherein the multiple time points comprise every week, every two weeks, every four weeks, every six weeks, or every eight weeks.

9. The method of any one of claims 3-8, wherein step (h) is performed at a time point after an anti-cancer treatment for the CNS cancer is administered to the subject.

10. The method of claim 9, wherein step (h) further comprises determining minimal residual disease (MRD) in the subject.

11. The method of claim 9, wherein the anti-cancer treatment comprises ionizing radiation, a chemotherapeutic agent, a therapeutic antibody, a checkpoint inhibitor, or any combination thereof.

12. The method of any one of claims 1-11, wherein the DNA sample comprises at least 0.1 ng of DNA.

13. The method of any one of claims 1-12, wherein the DNA sample comprises tumor derived DNA.

14. The method of any one of claims 1-13, wherein the DNA sample is from a cerebrospinal fluid sample.

15. The method of claim 14, wherein the DNA sample is obtained from the subject by lumbar puncture.

16. The method of any one of claims 1-13, wherein the DNA sample is from a blood plasma sample.

17. The method of claim 16, wherein the DNA sample is obtained from the subject by venipuncture.

18. The method of any one of claims 4-17, wherein an amplicon of the plurality of amplicons has a length of 100 basepairs or less.

19. The method of any one of claims 4-18, wherein an amplicon of the plurality of amplicons has a length of 200 basepairs or less.

20. The method of any one of claims 4-19, wherein the plurality of amplicons comprise nucleic acid sequences that can be mapped to a plurality of chromosomes.

21. The method of any one of claims 1-20, wherein the CNS cancer is meningioma, pituitary adenoma, craniopharyngioma, neurofibroma, hemangioblastoma, encephalocele, fibrous dysplasia, glioma, astrocytomas, oligodendrogliomas, glioblastomas, ependymal tumors, hemangiopericytoma, germ cell tumors, chordoma, chondrosarcoma, medulloblastoma, olfactory neuroblastoma, lymphoma, gliosarcoma, rhabdomyosarcoma, paranasal sinus cancer, or atypical teratoid/rhabdoid tumor (AT/RT).

22. The method of any one of claims 1-21, wherein the CNS cancer is glioblastoma (GBM), medulloblastoma, parenchymal metastases (PM), leptomeningeal disease (LMD), diffuse large B-cell lymphoma, or CNS lymphoma.

23. The method of any one of claims 1-22, wherein the obtaining step (a) comprises obtaining a first DNA sample and a second DNA sample from the subject.

24. The method of claim 23, wherein the first DNA sample is a cerebrospinal fluid sample.

25. The method of claim 23, wherein the second DNA sample is a blood plasma sample.

26. A method of treating a CNS cancer in a subject in need thereof, the method comprising:
(a) diagnosing the subject as having the CNS cancer according to any one of the claims 1-25; and
(b) administering an anti-cancer treatment to the subject.

27. The method of claim 26, wherein the CNS cancer is meningioma, pituitary adenoma, craniopharyngioma, neurofibroma, hemangioblastoma, encephalocele, fibrous dysplasia, glioma, astrocytomas, oligodendrogliomas, glioblastomas, ependymal tumors, hemangiopericytoma, germ cell tumors, chordoma, chondrosarcoma, medulloblastoma, olfactory neuroblastoma, lymphoma, gliosarcoma, rhabdomyosarcoma, paranasal sinus cancer, or atypical teratoid/rhabdoid tumor (AT/RT).

28. The method of claim 26 or 27, wherein the CNS cancer is glioblastoma (GBM), medulloblastoma, parenchymal metastases (PM), leptomeningeal disease (LMD), diffuse large B-cell lymphoma, or CNS lymphoma.

29. The method of any one of claims 26-28, wherein the anti-cancer treatment comprises ionizing radiation, a chemotherapeutic agent, a therapeutic antibody, a checkpoint inhibitor, or any combination thereof.
